(19) United States
(12) Patent Application Publication
(10) Pub. No.: US 2025/0090795 A1
(43) Pub. Date: Mar. 20, 2025

(54) SLEEP MANAGEMENT SYSTEM AND METHOD FOR CONTROLLING THE SAME

(71) Applicant: SAMSUNG ELECTRONICS CO., LTD., Suwon-si (KR)

(72) Inventors: Junghwi EUN, Suwon-si (KR); Chanwon LEE, Chanwon (KR)

(73) Assignee: SAMSUNG ELECTRONICS CO., LTD., Suwon-si (KR)

(21) Appl. No.: 18/925,960

(22) Filed: Oct. 24, 2024

Related U.S. Application Data
(63) Continuation of application No. PCT/KR2024/012840, filed on Aug. 28, 2024.

(30) Foreign Application Priority Data
Sep. 18, 2023 (KR) 10-2023-0124234

(51) Int. Cl. A61M 21/02

(52) U.S. Cl. CPC A61M 21/02; A61M 2205/3303; A61M 2205/3561

(57) ABSTRACT

Disclosed is a sleep management system including at least one sensor that to detects a biometric signal of a user, at least one electronic device, and a hub device that communicates with the at least one sensor and the at least one electronic device. The hub device identifies, based on the biometric signal of the user, a stage as a sleep preparation stage, a sleeping stage, or an awake stage, and transmits, to the at least one electronic device, a control command to control the at least one electronic device based on the identified stage.

### CROSS REFERENCE TO RELATED APPLICATIONS

This application is a bypass continuation application of International Application No. PCT/KR2024/012840, filed on Aug. 28, 2024, which is based on and claims priority to Korean Patent Application No. 10-2023-0124234, filed on Sep. 18, 2023, in the Korean Patent Office, the disclosures of which are incorporated by reference herein in their entireties.

### BACKGROUND

### 1. Field

The disclosure relates to a sleep management system that controls electronic devices based on the user's sleep state, and method for controlling the same.

### 2. Description of Related Art

People read books or watch contents such as e-books, music, movies or dramas before going to sleep. They use an electronic device such as a television (TV) to watch the contents. They may fall asleep without turning off the electronic device while watching the contents before going to sleep or may fall asleep without turning off a lighting device.

When the people fall asleep without turning off the electronic device, their bodies are tensed due to electromagnetic waves, heat, light, etc., which are produced from the electronic device. This causes the people to be unable to have a good night's sleep.

Furthermore, in a humid and hot night in the summer, people experience inconvenience because they need to wake up from their sleep and turn on the air conditioner and then turn off the air conditioner after getting up.

### SUMMARY

The disclosure relates to a sleep management system and method for controlling the same, which controls operations of electronic devices in response to a user's current stage related to sleep being a sleep preparation stage, a sleeping stage or an awake stage.

According to an aspect of the disclosure, there is provided a sleep management system including: at least one sensor configured to detect a biometric signal of a user; at least one electronic device; and a hub device configured to: communicate with the at least one sensor and the at least one electronic device; identify, based on the biometric signal of the user, a stage as a sleep preparation stage, a sleeping stage, or an awake stage; and transmit, to the at least one electronic device, a control command to control the at least one electronic device based on the identified stage.

The hub device may be configured to, based on the identified stage being the sleep preparation stage, transmit at least one of a command to close an automatic curtain, a humidity control command, a temperature control command, or a light-off command.

The hub device may be configured to, based on the identified stage being the sleeping stage, transmit at least one of a command to turn off a television, a command to turn off a projector, or a command to turn off a soundbar.

The hub device may be configured to, based on the identified stage being the awake stage, transmit at least one of a command to open an automatic curtain, a command to stop humidity control, a command to stop temperature control, and a light control command.

The hub device may be configured to, based on the identified stage being the awake stage, communicate with a user device, receive schedule information from the user device, and transmit a command to output the received schedule information.

The at least one sensor may include at least one of an image sensor, a microphone, a radar sensor, a pressure sensor, a temperature sensor, an electrocardiogram (ECG) sensor, an acceleration sensor, or a heart rate sensor, wherein the at least one electronic device may include at least one of an automatic curtain open/close device, a television, a projector, a soundbar, a lighting device, an air conditioner, or a humidifier.

The hub device may include an input interface; and a processor configured to generate a control command for each of the at least one electronic device based on target value information for each of the at least one electronic device received through the input interface.

The at least one sensor may be arranged at a bed placed in a room or a user device, and the at least one sensor or the user device may include a communicator for communicating with the hub device.

The hub device may include: an input interface; and a processor configured to identify the awake stage based on the biometric signal and awake time information received through the input interface.

According to an aspect of the disclosure, there is provided a method of controlling a sleep management system including: detecting, using at least one sensor, a biometric signal of a user; identifying, through a hub device based on the biometric signal of the user, a stage as a sleep preparation stage, a sleeping stage or an awake stage; and transmitting a control command to control at least one electronic device based on the identified stage to the at least one electronic device, wherein the at least one electronic device is configured to control an operation based on the control command.

The transmitting of the control command may include transmitting at least one of a command to close an automatic curtain, a humidity control command, a temperature control command, or a light off command based on the determining as the sleep preparation stage, wherein the transmitting of the control command may include, based on the identified stage being the sleep preparation stage, transmitting a command to play a sleep inducing content.

The transmitting of the control command may include, based on the identified stage being the sleeping stage, transmitting at least one of a command to turn off a television, a command to turn off a projector, or a command to turn off a soundbar.

The transmitting of the control command may include transmitting, based on the identified stage being the awake stage, at least one of a command to open an automatic curtain, a command to stop humidity control, a command to stop temperature control, or a light on command.

The transmitting of the control command may include transmitting, based on the identified stage being the awake stage, at least one of a television on command, a channel command and a volume command.

The transmitting of the control command may include, based on the identified stage being the awake stage, communicating with a user device, receiving schedule information from the user device, and transmitting a command to output the received schedule information.

According to the disclosure, the user may be induced to have a sound sleep and get up easily by controlling operations of electronic devices in response to a user's current stage related to sleep being a sleep preparation stage, a sleeping stage or an awake stage.

According to the disclosure, sleep disturbance due to an electronic device may be prevented by reducing brightness and sound volume of the electronic device when the user is at the sleep preparation stage. According to the disclosure, the sleep quality of the user may be improved by changing an unfit sleep environment to a sleep inducing environment.

According to the disclosure, power consumption of an electronic device may be reduced by powering off a lighting device, a TV, or the like when the user is at the sleeping stage.

According to the disclosure, a period of time to awake from sleep may be shortened and the user's condition may get better by controlling video, audio, lighting, temperature and humidity when the user is at the awake stage.

According to the disclosure, an optimized sleep environment may be provided for each user by controlling an electronic device for each of a sleep preparation stage, a sleeping stage and an awake stage based on control information for the electronic device configured for each user. For example, according to the disclosure, brightness of light may be controlled for each room where the user falls asleep or temperature and humidity of the room may be controlled.

### BRIEF DESCRIPTION OF THE DRAWINGS

The above and other aspects and/or features of one or more embodiments of the disclosure will be more apparent from the following description taken in conjunction with the accompanying drawings, in which:

FIG. 1 illustrates an example of a network of a sleep management system, according to an embodiment;

FIG. 2 schematically illustrates an example of a structure of a sleep management system, according to an embodiment;

FIG. 3 illustrates an example of a control block diagram of a sleep management system, according to an embodiment;

FIGS. 4 and 5 illustrate an example of a plurality of sensors of a sleep management system, according to one or more embodiments;

FIG. 6 illustrates an example of a flowchart of a sleep management system, according to an embodiment;

FIG. 7 is a diagram for describing a procedure for processing data collected from a plurality of sensors of a sleep management system, according to an embodiment;

FIG. 8 is a diagram for describing sleep stages, according to an embodiment;

FIG. 9 illustrates an example of sleep state information derived by a sleep management system, according to an embodiment;

FIG. 10 illustrates an example of sleep summary information derived by a sleep management system, according to an embodiment;

FIGS. 11 and 12 illustrate an example of a flowchart of a sleep management method, according to one or more embodiments;

FIG. 13 illustrates an example of a preset operation to induce sleep of the user, according to an embodiment;

FIG. 14 illustrates an example of a preset operation to relieve stress of the user, according to an embodiment;

FIG. 15 illustrates an example of a preset operation to relieve sleep disorders of the user, according to an embodiment;

FIG. 16 illustrates an example of a preset operation to induce wakeup of the user, according to an embodiment;

FIG. 17 is a configuration diagram of a sleep management system, according to an embodiment;

FIG. 18 is a control block diagram of a hub device and electronic devices in a sleep management system, according to an embodiment;

FIG. 19 is a control flowchart of an electronic device in a sleep management system, according to an embodiment;

FIG. 20 illustrates layouts of electronic devices at home managed by a sleep management system, according to an embodiment;

FIG. 21 illustrates control information for electronic devices for each stage configured by a sleep management system, according to an embodiment; and

FIGS. 22, 23 and 24 illustrate sleep environments of a room managed by a sleep management system, according to one or more embodiments.

### DETAILED DESCRIPTION

It should be understood that various embodiments of the disclosure and associated terms are not intended to limit technical features herein to particular embodiments, but encompass various changes, equivalents, or substitutions.

Like reference numerals may be used for like or related elements throughout the drawings.

The singular form of a noun corresponding to an item may include one or more items unless the context states otherwise.

Throughout the disclosure, “A or B”, “at least one of A and B”, “at least one of A or B”, “A, B or C”, “at least one of A, B and C”, and “at least one of A, B, or C” may each include any one or all the possible combinations of A, B and C.

The expression “and/or” is interpreted to include a combination or any of associated elements.

For example, the expression “A, B and/or C” may include one of A, B, and C or any combination thereof.

Terms like “first”, “second”, etc., may be simply used to distinguish an element from another, without limiting the elements in a certain sense (e.g., in terms of importance or order).

When an element is mentioned as being “coupled” or “connected” to another element with or without an adverb “functionally” or “operatively”, it means that the element may be connected to the other element directly (e.g., wired), wirelessly, or through a third element.

It will be further understood that the terms “comprise” and/or “comprising,” when used in this disclosure, specify the presence of stated features, integers, steps, operations, elements, parts or combinations thereof, but do not preclude the possible presence or addition of one or more other features, integers, steps, operations, elements, components, and/or groups thereof.

When an element is mentioned as being “connected to”, “coupled to”, “supported on” or “contacting” another element, it includes not only a case that the elements are directly connected to, coupled to, supported on or contact each other but also a case that the elements are connected to, coupled to, supported on or contact each other through a third element.

Throughout the disclosure, when an element is mentioned as being located “on” another element, it may imply that the element is abut on the other element or that a third element exists between the two elements.

A sleep management system according to various embodiments will now be described in detail in connection with the accompanying drawings.

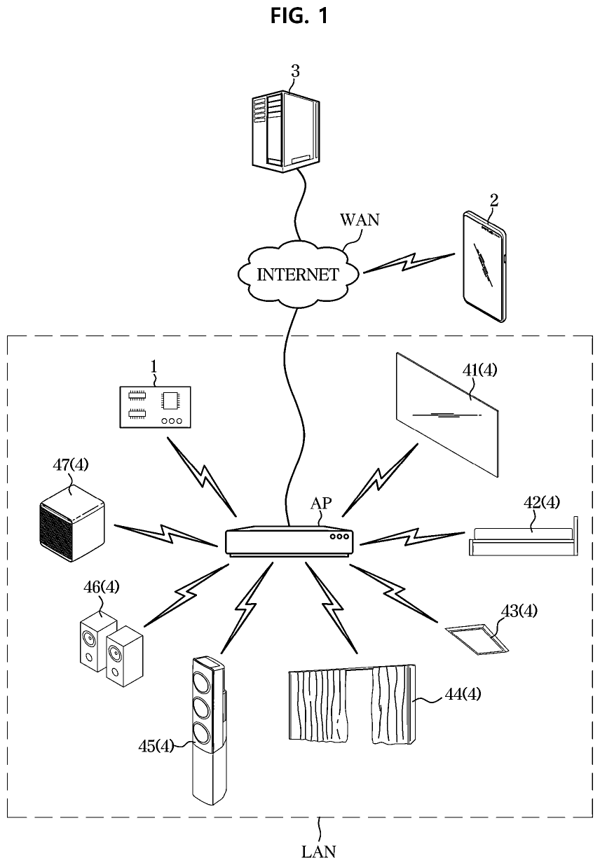

FIG. 1 illustrates an example of a network of a sleep management system, according to an embodiment.

Referring to FIG. 1, the network system according to an embodiment may include a hub device 1, a user device 2, a server device 3 and/or home appliances 4.

The hub device 1 may include a communication module capable of communicating with the user device 2, the server device 3 and/or the home appliances 4, at least one processor for processing data, and at least one memory that stores a program for controlling operation of the hub device 1.

The hub device 1 may obtain processed data based on processing of data collected from a plurality of sensors. In an embodiment, the hub device 1 may use a machine learning model to process the data collected from the plurality of sensors.

In an embodiment, the hub device 1 may transmit the processed data to the user device 2. For example, the hub device 1 may transmit the processed data to the user device 2 not through the server device 3 but by direct communication.

The home appliances 4 may include various types of electronic products. For example, the home appliances 4 may include at least one of a display device 41, a furniture control device 42, a lighting device 43, an automatic curtain open/close device 44, an air conditioner 45, a speaker 46 and an air purifier 47. The aforementioned home appliances are merely examples, and other various types of electronic products such as a clothes care apparatus in addition to the aforementioned home appliance products may also be included in the home appliances 4.

The home appliance 4 may be controlled remotely by the server device 3.

The furniture control device 42 may include an actuator that may change a posture of the user by changing the structure of the furniture and/or a vibration element that may transmit vibration to the user who lies or sits on the furniture. For example, the furniture control device 42 may include an actuator that is able to control a reclination angle of a recliner bed, a recliner chair and/or a recliner sofa.

The lighting device 43 may include a light source with a controllable intensity and/or color of light.

The automatic curtain open/close device 44 may include an actuator for automatically opening or closing a curtain.

The server device 3 may include a communication module for communicating with the hub device 1, the user device 2 and/or the home appliance 4.

The server device 3 may include at least one processor that may process data received from the hub device 1, the user device 2 and/or the home appliances 4, and at least one memory that may store a program for processing data or processed data. The server device 3 may be implemented with various computing devices such as a workstation, a cloud, a data drive, a data station, etc. The server device 3 may be implemented with one or more servers physically or logically classified based on function, sub-configuration of the function or data, and may transmit or receive data through inter-server communication and process the data.

The server device 3 may perform functions of storing and/or managing a user account, registering the hub device 1, the user device 2 and/or the home appliance 4 by associating them with the user account, and managing or controlling the registered hub device 1 and the home appliance 4. For example, the user may access the server device 3 through the user device 2 to create a user account. The user account may be identified by an identity (ID) and a password created by the user. The user may access the server device 3 through the user device 2 to manage the user account. The server device 3 may register the hub device 1, the user device 2 and/or the home appliance 4 with the user account, according to a set procedure. For example, the server device 3 may connect identification information (e.g., a serial number, a media access control (MAC) address, etc.) of the hub device 1 to the user account to register, manage and control the hub device 1. Likewise, the server device 3 may register the user device 2 and the home appliance 4 with the user account and control them.

The server device 3 may receive various information from the hub device 1, the user device 2 and/or the home appliance 4 registered with the user account.

For example, the server device 3 may include a first server and a second server. The first server may create and/or manage user account information, and register and/or manage information about the hub device 1, the user device 2 and/or the home appliance 4 with the user account. The second server may receive registration information of the user device 2 and the home appliance 4 from the first server to control the user device 2 and/or the home appliance 4.

In another example, the second server may perform a function of managing the hub device 1 and the home appliance 4 registered in the first server on behalf of the first server.

The number of the server devices 3 is not limited thereto, and the server device 3 may include a plurality of servers for performing the same and/or different operations.

The user device 2 may include a communication module for communicating with the hub device 1, the server device 3 and/or the home appliance 4. The user device 2 may include a user interface for receiving user inputs or outputting information for the user. The user device 2 may include at least one processor for controlling operation of the user device 2 and at least one memory for storing a program for controlling the operation of the user device 2.

The user device 2 may be carried by the user or placed at the user's home or office. The user device 2 may include a personal computer, a terminal, a mobile phone, a smart phone, a handheld device, a wearable device, a display device, etc., without being limited thereto.

In the memory of the user device 2, a program, i.e., an application for processing data received from the hub device 1 may be stored. The application may be sold in a state of being installed in the user device 2, or may be downloaded and installed from an external server.

The user may access the server device 3 and create a user account by running the application installed in the user device 2, and register the hub device 1 and/or the home appliance 4 by communicating with the server device 3 based on the login user account.

For example, when the home appliance 4 is operated to access the server device 3 according to a procedure guided in the application installed in the user device 2, the server device 3 may register the home appliance 4 with the user account by registering the identification information (e.g., a serial number or a MAC address) of the home appliance 4 with the user account. The home appliance 4 may also be registered with the user account in the similar manner. It is obvious that other information than the serial number or MAC address of the device to identify the device may be used for the information required to register the device such as the home appliance 4 with the user account.

The user device 2 may receive various information from the hub device 1 and the home appliance 4 registered with the user account directly or through the server device 3.

A network may include both a wired network and a wireless network. The wired network may include a cable network or a telephone network, and the wireless network may include any network that transmits or receives signals in radio waves. The wired network and the wireless network may be connected to each other.

The network may include a wide area network (WAN) such as the Internet, a local area network (LAN) formed around an access point (AP), and a short-range wireless network without an AP. The short-range wireless network may include bluetooth (IEEE 802.15.1), Zigbee (IEEE 802.15.4), wireless fidelity (Wi-Fi) direct, near field communication (NFC), Z-wave, etc., without being limited thereto.

The AP may connect the hub device 1, the user device 2 and/or the home appliance 4 to the WAN connected to the server device 3. The hub device 1, the user device 2 and/or the home appliance 4 may be connected to the server device 3 through the WAN.

The AP may use wireless communication such as Wi-Fi (IEEE 802.11), Bluetooth (IEEE 802.15.1), Zigbee (IEEE 802.15.4), etc., to communicate with the hub device 1, the user device 2 and/or the home appliance 4, and use wired communication to access the WAN, but the wireless communication scheme of the AP is not limited thereto.

In an embodiment, the hub device 1 may communicate with the user device 2 over a short-range wireless network without going through the AP.

For example, the hub device 1 may be connected to the user device 2 over a short-range wireless network (e.g., Wi-Fi direct, bluetooth or NFC). In another example, the hub device 1 may use the long-range wireless network (e.g., a cellular communication module) to be connected to the user device 2 through the WAN.

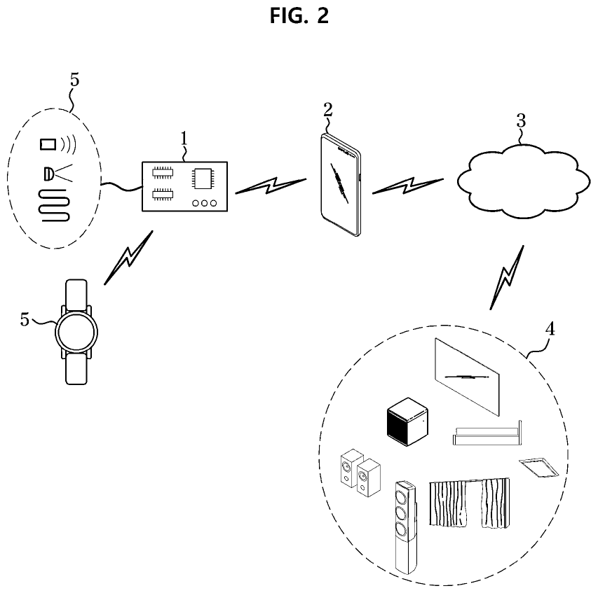

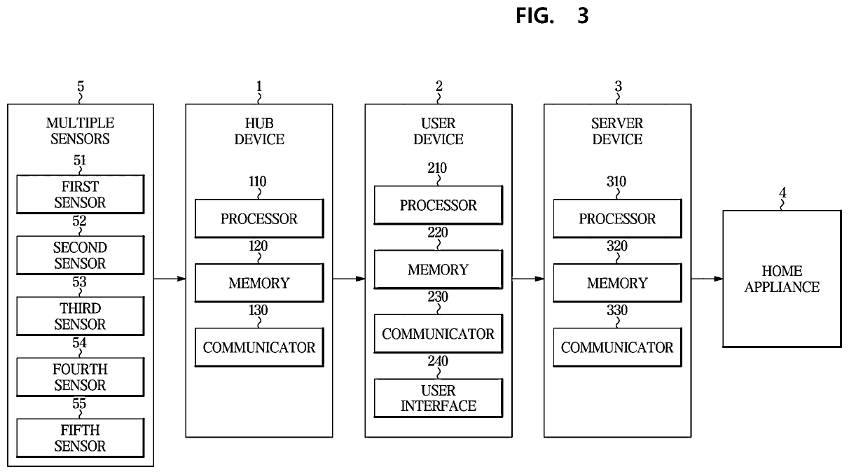

FIG. 2 schematically illustrates an example of a structure of a sleep management system, according to an embodiment. FIG. 3 illustrates an example of a control block diagram of a sleep management system, according to an embodiment.

Referring to FIGS. 2 to 3, the hub device 1 may receive data collected from a plurality of sensors 5.

The plurality of sensors 5 may include sensors (e.g., a first sensor 51, a second sensor 52, a third sensor 53, a fourth sensor 54 and/or a fifth sensor 55) for collecting data of the user.

The plurality of sensors 5 may each collect data of the user and transmit the collected data of the user to the hub device 1.

In an embodiment, the data of the user may include data related to the user's sleep.

The data related to the user's sleep may include pressure data for measuring a pressure change corresponding to a change in posture of the user, displacement data corresponding to displacement of the body that changes according to the user's breathing, oxygen saturation data corresponding to the user's oxygen saturation, electrocardiogram data corresponding to the user's electrocardiogram, acceleration data corresponding to acceleration that changes according to the user's movement, and/or eye-movement data corresponding to the movement of the user's eyes.

The plurality of sensors 5 may include at least two of a pressure sensor for collecting pressure data for measuring a pressure change corresponding to a change in the user's posture, a ultra-wideband (UWB) sensor for measuring displacement data corresponding to displacement of the body that changes according to the user's breathing, an oxygen saturation sensor for collecting oxygen saturation data corresponding to the user's oxygen saturation, an electrocardiogram sensor for collecting electrocardiogram data corresponding to the user's electrocardiogram, an acceleration sensor for collecting acceleration data corresponding to acceleration that changes according to the user's movement, and/or a radar sensor for collecting eye-movement data corresponding to the user's eye movement.

The terms first, second, third, fourth and fifth from the expressions, the first sensor 51, the second sensor 52, the third sensor 53, the fourth sensor 54 and the fifth sensor 55 indicate that the respective sensors are different sensors.

Each of the first sensor 51, the second sensor 52, the third sensor 53, the fourth sensor 54 and the fifth sensor 55 may be one of the pressure sensor, the UWB sensor, the radar sensor, the oxygen saturation sensor, the electrocardiogram sensor or the acceleration sensor.

In the following description, for convenience of explanation, the first sensor 51 is defined as the pressure sensor, the second sensor 52 the UWB sensor, the third sensor 53 the radar sensor, the fourth sensor 54 the oxygen saturation sensor and/or the electrocardiogram sensor, and the fifth sensor 55 the acceleration sensor.

The plurality of sensors 5 may further include an extra sensor (e.g., a mike or camera) in addition to the first to fifth sensors 51 to 55, or may not include at least one of the first to fifth sensors 51 to 55.

Data collected from the plurality of sensors 5 may be sent to the hub device 1.

In an embodiment, data collected by at least one of the plurality of sensors 5 may be sent to the hub device 1 by wired communication, and data collected by the other sensor(s) may be sent to the hub device 1 by wireless communication.

Accordingly, the hub device 1 may be wiredly connected to at least one of the plurality of sensors 5 and wirelessly connected to the other sensor(s).

In an embodiment, the fourth sensor 54 may be included in a smart sensor device (e.g., a wearable device). The smart sensor device may include a wireless communication module and the fourth sensor 54. For example, the smart sensor device may include a smart watch that is shaped like a watch and/or a smart ring that is shaped like a ring, but the form of the smart sensor device is not limited thereto.

The smart sensor device may establish wireless communication with the hub device 1, and transmit data collected from the fourth sensor 54 to the hub device 1 by wireless communication.

In an embodiment, the smart sensor device may include the fourth sensor 54 and the fifth sensor 5. The smart sensor device may establish wireless communication with the hub device 1, and transmit data collected from the fourth sensor 54 and the fifth sensor 55 to the hub device 1 by wireless communication.

In an embodiment, the data collected from the plurality of sensors 5 may be sent to the hub device 1 by wired communication.

In various embodiments, the plurality of sensors 5 may include an imaging sensor (e.g., a camera). However, in an embodiment, the plurality of sensors 5 may all be non-imaging sensors.

In an embodiment of the disclosure, as all the plurality of sensors 5 correspond to non-imaging sensors, an invasion of the user's privacy may be prevented.

In an embodiment, the hub device 1 may include at least one memory 120 for storing a program for processing data collected from the plurality of sensors 5, and at least one processor 110 that is able to process the data collected from the plurality of sensors 5 based on the program stored in the at least one memory 120.

The at least one memory 120 may store a machine learning model for processing the data collected from the plurality of sensors 5.

In an embodiment, the machine learning model may be one for feature extraction, which extracts a feature of the data collected from the plurality of sensors 5 when the data is input thereto, and outputs processed data including the extracted feature.

The feature of the data may include elements extracted from the data by the machine learning model to perform classification or prediction.

In an embodiment, the machine learning model may be one for sleep stage decision, which outputs processed data including data of a sleep stage of the user when the data collected from the plurality of sensors 5 is input thereto.

For example, the at least one processor 110 may include a first machine learning model 11 for processing first data collected from the first sensor 51, a second machine learning model 12 for processing second data collected from the second sensor 52, a third machine learning model 13 for processing third data collected from the third sensor 53, a fourth machine learning model 14 for processing fourth data collected from the fourth sensor 54, and a fifth machine learning model 15 for processing fifth data collected from the fifth sensor 55.

The at least one processor 110 may obtain first processed data based on processing of the first data collected from the first sensor 51.

The first processed data may include feature data extracted from the first data and/or data about a sleep stage extracted from the first data. The volume of the first processed data may be smaller than the volume of the first data.

The at least one processor 110 may obtain second processed data based on processing of the second data collected from the second sensor 52.

The second processed data may include feature data extracted from the second data and/or data about a sleep stage extracted from the second data. The volume of the second processed data may be smaller than the volume of the second data.

The at least one processor 110 may obtain third processed data based on processing of the third data collected from the third sensor 53.

The third processed data may include feature data extracted from the third data and/or data about a sleep stage extracted from the third data. The volume of the third processed data may be smaller than the volume of the third data.

The at least one processor 110 may obtain fourth processed data based on processing of the fourth data collected from the fourth sensor 54.

The fourth processed data may include feature data extracted from the fourth data and/or data about a sleep stage extracted from the fourth data. The volume of the fourth processed data may be smaller than the volume of the fourth data.

The at least one processor 110 may obtain fifth processed data based on processing of the fifth data collected from the fifth sensor 55.

The fifth processed data may include feature data extracted from the fifth data and/or data about a sleep stage extracted from the fifth data. The volume of the fifth processed data may be smaller than the volume of the fifth data.

In the disclosure, the hub device 1 primarily processes data and transmits it to the user device 2, thereby reducing data throughput to be borne by the user device 2.

The hub device 1 may include a communicator 130 including a wired communication module for performing wired communication with the plurality of sensors 5, and/or a wireless communication module for performing wireless communication with the user device 2, the server device 3 and/or the home appliances.

The hub device 1 may include a printed circuit board (PCB) including the at least one processor 110, the at least one memory 120 and the communicator 130. At least some of the plurality of sensors 5 may be wiredly connected to the PCB.

The hub device 1 may include a housing that covers the PCB.

The hub device 1 is likely to be installed in a location where user operation is difficult. Hence, in an embodiment, the hub device 1 may not include any user interface device (input/output device).

In preparation for an occasion when the hub device 1 is installed in a place where user may easily operate the hub device 1, the hub device 1 may include a user interface device (input/output device) in an embodiment.

In an embodiment, the user may operate the user interface device configured in the hub device 1 to connect the hub device 1 to an AP.

In an embodiment, the user may operate the user interface device configured in the hub device 1 to activate the communicator 130 of the hub device 1.

In an embodiment, the user may operate the user interface device configured in the hub device 1 to power on the hub device 1.

The at least one processor 110 may control the plurality of sensors 5.

For example, the at least one processor 110 may control at least one of the plurality of sensors 5 connected via wires.

The at least one processor 110 may wake up at least one of the plurality of sensors 5 connected via wires based on a sensor wakeup condition being satisfied.

The waking up of the sensor may include activating the sensor.

The at least one processor 110 may switch at least one of the plurality of sensors 5 connected via wires into a standby state based on a sensor standby condition being satisfied. The switching of the sensor into the standby state may include inactivating the sensor or driving the sensor in a low power mode.

The user device 2 may receive, from the hub device 1, data obtained by the hub device 1 by wireless communication.

The data obtained by the hub device 1 may include processed data resulting from processing of the data collected from the plurality of sensors 5.

In an embodiment, the user device 2 may include at least one memory 220 for storing a program for processing data received from the hub device 1, and at least one processor 210 that is able to process the data received from the hub device 1 based on the program stored in the at least one memory 220.

The at least one memory 220 may store a machine learning model for processing the data received from the hub device 1.

The at least one memory 220 may store a sleep management application that is downloadable from an external server. The sleep management application may be a downloadable app, at least a portion of which may be at least temporarily stored or arbitrarily created in a recording medium that may be readable to a device such as a server of the manufacturer, a server of the application store, or a relay server.

The sleep management application may include a machine learning model. The machine learning model included in the sleep management application may be updated by the external server.

In an embodiment, the user device 2 may include a communicator 230 including at least one communication module for establishing communication with the hub device 1, the server device 3, the home appliance 4 and/or the smart sensor device including at least some of the plurality of sensors 5.

The user device 2 may receive the processed data from the hub device 1 through the communicator 230.

In an embodiment, the at least one processor 210 may establish communication between the communicator 130 of the hub device 1 and the communicator 230 (e.g., a short-range wireless communication module) of the user device 2 in response to the communicator 230 being activated.

In an embodiment, the at least one processor 210 may control a user interface 240 to provide feedback that requests activation of the communicator 230 in response to the sleep management application being executed while the communicator 230 (e.g., the short-range wireless communication module) is not activated.

The at least one processor 210 may wirelessly receive data from the hub device 1 through the communicator 230 (e.g., the short-range wireless communication module).

The at least one processor 210 may use the machine learning model stored in the at least one memory 220 to process the data received from the hub device 1.

The data received from the hub device 1 may include the first processed data, the second processed data, the third processed data, the fourth processed data and/or the fifth processed data.

The at least one processor 210 may obtain sleep state information relating to a sleep state of the user by inputting the first processed data, the second processed data, the third processed data, the fourth processed data and/or the fifth processed data to the machine learning model stored in the at least one memory 220.

The sleep state information relating to the user's sleep state may include at least one of information about the user's sleep stage, information about the user's stress index, or information about the user's sleep disorder.

The machine learning model stored in the at least one memory 220 may be one for decision of the user's sleep state, which outputs sleep state information relating to the user's sleep state when the first processed data, the second processed data, the third processed data, the fourth processed data and/or the fifth processed data are input thereto.

The at least one processor 210 may store the sleep state information obtained by inputting the first processed data, the second processed data, the third processed data, the fourth processed data and/or the fifth processed data to the machine learning model in the at least one memory 220.

In an embodiment, the at least one processor 210 may generate sleep summary information based on the sleep state information accumulated and stored in the at least one memory 220. The at least one processor 210 may control the user interface 240 to output sensory information corresponding to the sleep summary information based on a preset condition being satisfied (e.g., based on receiving of a user input to check the sleep summary information).

The at least one processor 210 may control the communicator 230 to transmit the sleep state information obtained by inputting the first processed data, the second processed data, the third processed data, the fourth processed data and/or the fifth processed data to the machine learning model.

The communicator 230 may include a first communication module for establishing communication with the hub device 1 and a second communication module for establishing communication with the server device 3. Accordingly, the communicator 230 may communicate with the hub device 1 in a first communication scheme and at the same time, communicate with the server device 3 in a second communication scheme.

In an embodiment of the disclosure, the sleep state information may be obtained after the data collected from the plurality of sensors 5 is primarily processed by the hub device 1 and secondarily processed by the user device 2, and the sleep state information may be transmitted to the server device 3 in real time.

In an embodiment of the disclosure, the data collected from the plurality of sensors 5 and associated with the user's privacy may not be sent directly to the server device 3.

The user device 2 may include the user interface 240 for communication with the user.

In various embodiments, it is also possible to obtain the sleep state information after the hub device 1 primarily processes the data collected from the plurality of sensors 5 and secondarily processes the primarily processed data.

In various embodiments, it is also possible to obtain the sleep state information after the hub device 1 primarily processes the data collected from the plurality of sensors 5 and transmits the primarily processed data to the server, and the server secondarily processes the primarily processed data.

In various embodiments, it is also possible to obtain the sleep state information after the hub device 1 transmits the data collected from the plurality of sensors 5 to the user device 2, and the user device 2 primarily processes the data received from the hub device 1 and secondarily processes the primarily processed data.

In various embodiments, it is also possible to obtain the sleep state information after the hub device 1 transmits the data collected from the plurality of sensors 5 to the server device 3 and the server device 3 primarily processes the data received from the hub device 1 and secondarily processes the primarily processed data.

The user interface 240 may obtain a user input. The user interface 240 may provide various information about operations of the user device 2. The user interface 240 may include an input interface and an output interface.

The input interface may convert the sensory information received from the user into an electric signal. The electric signal may correspond to a user input. The user input may include various commands. The input interface may transmit the electric signal (voltage or current) corresponding to the user input to the at least one processor 210.

The input interface may include various input devices to convert tactile information to an electric signal. For example, the input interface may be configured with a physical button or a touch screen. The input interface may include a mike to convert auditory information to an electric signal.

The input interface may receive a user input to run the sleep management application.

The output interface may output information relating to operations of the user device 2. The output interface may display information input by the user or information to be provided for the user in various screens. The output interface may display information regarding an operation of the user device 2 in at least one of an image or text. For example, the output interface may output an interface of the sleep management application. Furthermore, the output interface may display a graphic user interface (GUI) that enables the user device 2 to be controlled. In other words, the output interface may display a user interface element (UI element) such as an icon.

The output interface may output an interface corresponding to the sleep management application.

For example, the output interface may include a liquid crystal display (LCD) panel, light emitting diode (LED) panel, an organic LED (OLED) panel, or a micro LED panel. The output interface may include a touch display that serves as an input device as well.

The output interface and the input interface may be configured separately or in one device (e.g., the touch display).

The server device 3 may receive, from the user device 2, data obtained by the user device 2 by wireless communication.

The data obtained by the user device 2 may include the aforementioned sleep state information.

In an embodiment, the server device 3 may include at least one memory 320 for storing a program for processing the sleep state information received from the user device 2, and at least one processor 310 that is able to process the sleep state information received from the user device 2 based on the program stored in the at least one memory 320.

The at least one memory 320 may store the sleep state information received from the user device 2.

The at least one memory 320 may store a program for generating sleep summary information based on the sleep state information received from the user device 2.

The at least one processor 310 may generate the sleep summary information based on the sleep state information accumulated and stored in the at least one memory 320.

The at least one memory 320 may store a program for controlling the home appliance 4 based on the sleep state information received from the user device 2.

The at least one processor 310 may control the home appliance 4 based on the sleep state information received from the user device 2.

In various embodiments, the program for generating the sleep summary information based on the sleep state information and the program for controlling the home appliance 4 based on the sleep state information may be stored in different servers.

For example, a first server included in the server device 3 may store the program for generating the sleep summary information based on the sleep state information, and a second server included in the server device 3 may store the program for controlling the home appliance 4 based on the sleep state information.

The at least one memory 320 may store the sleep summary information based on the sleep state information.

In an embodiment, the server device 3 may include a communicator 330 including at least one communication module for establishing communication with the hub device 1, the user device 2, the home appliance 4 and/or the smart sensor device including at least some of the plurality of sensors 5.

The server device 3 may receive the sleep state information from the user device 2 through the communicator 330.

The server device 3 may transmit a control command to the home appliance 4 through the communicator 330.

The server device 3 may transmit the sleep summary information to the user device 2 through the communicator 330.

In an embodiment of the disclosure, as the server device 3 performs various operations based on the sleep state information, the user's sleep may be managed in various ways.

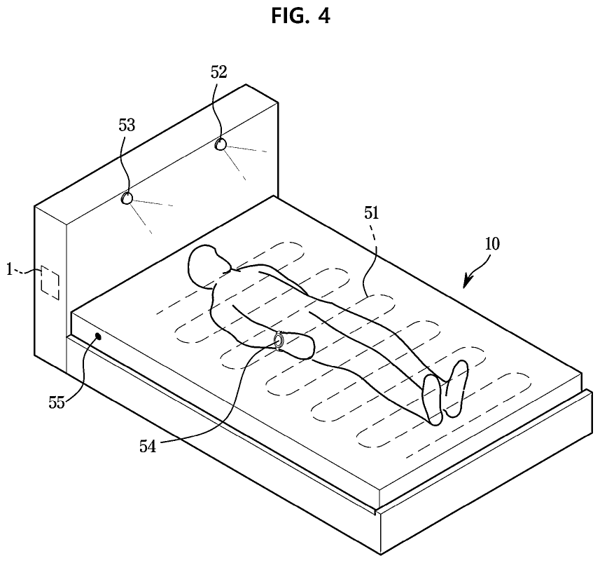

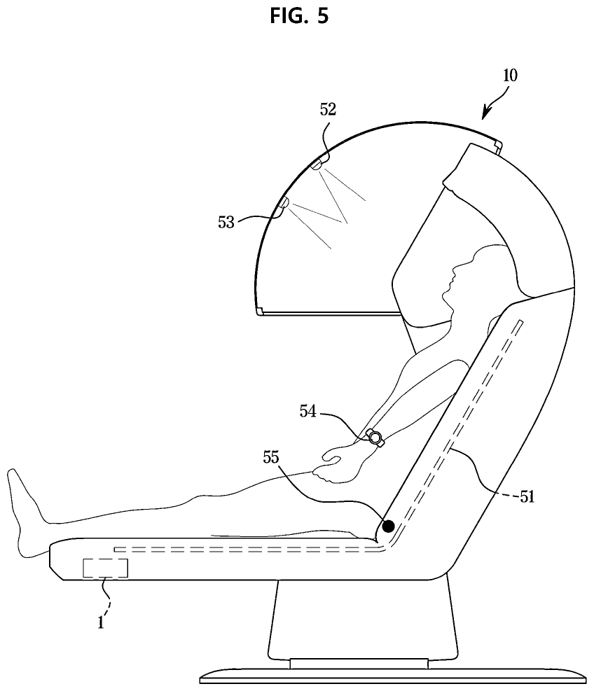

FIGS. 4 and 5 illustrate an example of a plurality of sensors of a sleep management system, according to an embodiment.

Referring to FIGS. 4 and 5, in an embodiment, some of the plurality of sensors 5 (e.g., the first sensor 51, the second sensor 52 and/or the third sensor 53) may be installed on a piece of furniture 10, and the others (e.g., the fourth sensor 54 and/or the fifth sensor 55) among the plurality of sensors 5 may be configured in the smart sensor device (e.g., a smart watch, a smart ring, etc.).

At least some of the plurality of sensors 5 may be configured on the furniture 10 on which the user may sit or lie.

The furniture 10 on which the user may sit or lie may include, for example, a bed, a chair and/or a sofa, but obviously, any furniture having the form that allows the user to sit or lie thereon may be used as the furniture 10 without limitation.

In an embodiment, the furniture 10 such as a bed, a chair and/or a sofa may include an actuator that is able to change the user's posture by changing its structure and/or a vibration element capable of transmitting vibration to the user.

The first sensor 51 may include a pressure sensor. The pressure sensor may include a piezoelectric element that generates an electric signal corresponding to displacement created by the pressure.

The first sensor 51 may be installed in a location where the pressure created by the user's body (e.g., the whole body) when the user lies or sits may be measured.

For example, when the furniture 10 corresponds to a bed, the first sensor 51 may be configured in a mattress where pressure occurs by the user's body. The mattress may include a cover having a polygonal or circular flat shape, defining the exterior and having an accommodation space, and a pad arranged in the accommodation space of the cover and including the first sensor 51. The mattress may be placed on the floor, a chair, a sofa or a bed.

The mattress may further include springs and/or sponge. The spring and/or the sponge may be arranged in the accommodation space of the cover.

The structure (e.g., length, layout, etc.) of the first sensor 51 may vary by the size of the mattress.

In another example, when the furniture 10 corresponds to a chair, the first sensor 51 may be configured in a seating portion, a backrest portion, a headrest and/or a leg portion where pressure occurs by the user's body.

The seating portion may include a portion coming into contact with the user's buttocks, the backrest portion may include a portion coming into contact with the user's back, the headrest may include a portion coming into contact with the user's head, and the leg portion may include a portion coming into contact with the user's legs.

The location of the first sensor 51 is not limited to the example shown in FIGS. 4 and 5, and the first sensor 51 may be installed at various locations where the pressure created by the body of the user who lies or sits on the furniture 10 may be measured.

The first sensor 51 may measure the pressure created by the user who lies or sits on the furniture 10. For example, the first sensor 51 may measure a distribution of the pressure that occurs by the user who lies or sits on the furniture 10. The first sensor 51 may obtain pressure data corresponding to the pressure created by the user who lies or sits on the furniture 10.

The second sensor 52 may include a UWB sensor. The UWB sensor may include a UWB signal irradiator for transmitting an ultra-wide band (UWB) signal and a UWB signal receiver for receiving a UWB signal reflected by the user's body.

The second sensor 52 may have a detection region facing the body (e.g., torso) of the user who lies or sits on the furniture 10. The second sensor 52 may have a detection region that may detect displacement of the body caused by the user's breathing. The second sensor 52 may be configured on the frame of the furniture 10 to have the detection region facing the body (e.g., torso) of the user, but the location of the second sensor 52 is not limited thereto.

For example, the second sensor 52 may have a detection region facing a portion of the body of the user who lies or sits on the furniture 10.

For example, when the furniture 10 corresponds to a bed, the second sensor 52 may have a detection region facing a center portion of the bed.

In another example, when the furniture 10 corresponds to a chair, the second sensor 52 may have a detection region facing a backrest portion of the chair.

The second sensor 52 may transmit a UWB signal to the body of the user and receive a UWB signal reflected from the body of the user.

The second sensor 52 may measure displacement of the user's body based on the UWB signal reflected from the body of the user. For example, the second sensor 52 may measure displacement of the user's body based on a time of flight (ToF) of the UWB signal. In another example, the second sensor 52 may use the Doppler effect to measure the displacement of the user's body according to a change in wavelength (and frequency) of the UWB signal.

In other words, the second sensor 52 may obtain displacement data corresponding to the displacement of the body that changes according to the user's breathing.

The third sensor 53 may include a radar sensor. The third sensor 53 may include a radar signal irradiator for transmitting a radar signal (e.g., millimeter waves or an mmWave signal) and a radar signal receiver for receiving a radar signal (e.g., an mmWave signal) reflected from the user's body.

A frequency band (e.g., 28 GHz) of the radar signal output from the third sensor 53 may be higher than the frequency band (e.g., 6.0 to 8.8 GHZ) of the UWB signal output from the second sensor 52.

A bandwidth of the radar signal output from the third sensor 53 may be narrower than the bandwidth of the UWB signal output from the second sensor 52.

The third sensor 3 may have a detection region facing the body (e.g., face) of the user who lies or sits on the furniture 10. The third sensor 53 may have a detection region that may detect a movement of eyes of the user. The third sensor 53 may be configured on the frame of the furniture 10 to have the detection region facing the user's body (e.g., torso), but the position of the third sensor 53 is not limited thereto.

The third sensor 53 may have a detection region facing a portion of the body of the user who lies or sits on the furniture 10.

For example, when the furniture 10 corresponds to a bed, the third sensor 53 may have a detection region facing a head area of the bed.

In another example, when the furniture 10 corresponds to a chair, the third sensor 53 may have a detection region facing the headrest of the chair.

The third sensor 53 may transmit a radar signal (mmWave signal) to the body of the user and receive an mmWave signal reflected from the body of the user.

The third sensor 53 may measure a movement of the eyes of the user based on the mmWave reflected from the eyes of the user.

In other words, the third sensor 53 may obtain eye-movement data corresponding to the movement of the eyes of the user.

The fourth sensor 54 may include an oxygen saturation sensor and/or an electrocardiogram sensor. The oxygen saturation sensor and/or the electrocardiogram sensor may include a light source for irradiating light and a photo receiver for receiving light reflected from the user's body.

The fourth sensor may have a detection region facing the body of the user who lies or sits on the furniture 10.

The fourth sensor 54 may be arranged in a smart sensor device (e.g., a smart watch, a smart ring, etc.) that may be worn by the user.

The fourth sensor 54 may operate in a non-invasive manner, irradiating light to a portion (e.g., a wrist) of the user's body and receiving light reflected from the user's body.

The fourth sensor 54 may measure an oxygen saturation level in the user's blood and an electrocardiogram (ECG) of the user based on the intensity of the light reflected from the user's body.

A portion of the light irradiated to a portion of the body may be absorbed in a blood vessel, and the oxygen saturation level in the user's blood or the user's ECG may be measured according to the light absorption rate and patterns of the absorbed light.

The fifth sensor 55 may include an acceleration sensor. The acceleration sensor may include a microelectromechanical system (MEMS) sensor, a 3-axis acceleration sensor and/or a 6-axis acceleration sensor.

In various embodiments, like the fourth sensor 54, the fifth sensor 55 may be configured in a smart sensor device (e.g., a smart watch, a smart ring, etc.) that may be worn by the user.

In an embodiment, the fifth sensor 55 may be installed on the furniture 10.

The fifth sensor 55 may obtain acceleration data corresponding to the movement of the user's body.

The hub device 1 may be installed on the furniture 10 or at a location adjacent to the furniture 10 and wiredly connected to some of the plurality of sensors 5. Furthermore, the hub device 1 may perform wireless communication with the smart sensor device (e.g., a wearable device) worn by the user.

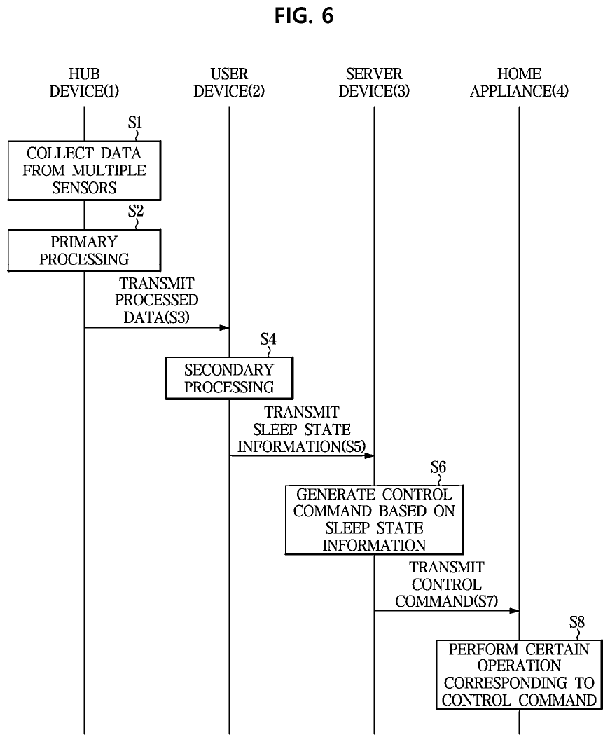

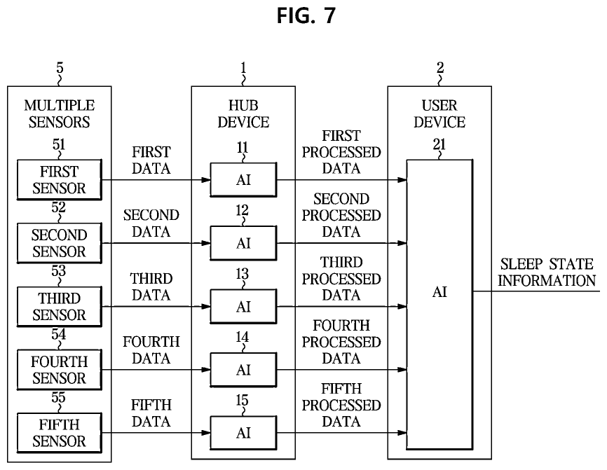

FIG. 6 illustrates an example of a flowchart of a sleep management system, according to an embodiment. FIG. 7 is a diagram for describing a procedure for processing data collected from a plurality of sensors of a sleep management system, according to an embodiment.

Referring to FIGS. 6 to 7, the hub device 1 may collect data from the plurality of sensors 5, in S1.

When the plurality of sensors 5 maintain in an active state, there may be a lot of consumption of power supplied to the plurality of sensors 5. The plurality of sensors 5 maintaining in the active state may include the plurality of sensor 5 maintaining in a state of obtaining sensor data by receiving power.

In an embodiment, the hub device 1 may maintain at least some of the plurality of sensors 5 in an inactive state and change the plurality of sensors 5 into an active state based on a preset condition being satisfied.

For example, the hub device 1 may determine whether there is a user on the furniture 10 based on processing of the data collected from the plurality of sensors 5. The presence of a user on the furniture 10 may include the user lying or sitting on the furniture 10.

The hub device 1 may switch the plurality of sensors 5 into a standby state based on determining that there is no user on the furniture 10.

For example, the hub device 1 may deactivate other sensors than the first sensor 51 and operate the first sensor 51 in a low power mode. The deactivating of the sensor may include blocking power supplied to the sensor.

The operating of the sensor in the low power mode may include setting an operation period (e.g., a data collection period) of the sensor to be longer.

The hub device 1 may determine whether there is the user on the furniture 10 based on processing of the data collected by the sensor (e.g., the first sensor 51) operating in the low power mode among the plurality of sensors 5.

The hub device 1 may wake up the plurality of sensors 5 based on determining that there is a user on the furniture 10.

Based on activating of the plurality of sensors 5, the collected data may be transmitted to the hub device 1.

The first sensor 51 may send the first data to the hub device 1, the second sensor 52 may send the second data to the hub device 1, the third sensor 53 may send the third data to the hub device 1, the fourth sensor 54 may send the fourth data to the hub device 1, and the fifth sensor 55 may send the fifth data to the hub device 1.

In an embodiment, at least some of the plurality of sensors 5 may send the sensor data to the hub device 1 by wired communication, and the others of the plurality of sensors 5 may send the sensor data to the hub device 1 by wireless communication.

The hub device 1 may primarily process the data collected from the plurality of sensors 5, in S2. For this, the hub device 1 may be equipped with a machine learning model.

The data collected from the plurality of sensors 5 may be processed by the machine learning models 11, 12, 13, 14 and 15 installed in the hub device 1.

The first machine learning model 11 for extracting a feature from the first data collected from the first sensor 51, a second machine learning model 12 for extracting a feature from the second data collected from the second sensor 52, a third machine learning model 13 for extracting a feature from the third data collected from the third sensor 53, a fourth machine learning model 14 for extracting a feature from the fourth data collected from the fourth sensor 54, and a fifth machine learning model 15 for extracting a feature from the fifth data collected from the fifth sensor 55 may be installed in the hub device 1.

The first machine learning model 11 may be pre-trained to extract a feature from pressure data collected by the pressure sensor. The second machine learning model 12 may be pre-trained to extract a feature from displacement data collected by the UWB sensor. The third machine learning model 13 may be pre-trained to extract a feature from eye-movement data collected by the radar sensor. The fourth machine learning model 14 may be pre-trained to extract a feature from oxygen saturation data and/or ECG data collected by the oxygen saturation sensor and/or the ECG sensor. The fifth machine learning model 15 may be pre-trained to extract a feature from acceleration data collected by the acceleration sensor.

The first machine learning model 11 may use the first data collected by the first sensor 51 as input data to output the first processed data as output data. The first processed data may include, for example, information about the user's posture, respiration rate and heart rate inferred by the first data. In another example, the first processed data may include information about the user's sleep stage, information about the user's stress index, or information about the user's sleep disorder inferred by the first data.

The second machine learning model 12 may use the second data collected by the second sensor 52 as input data to output the second processed data as output data. The second processed data may include, for example, information about a respiration rate and a heart rate inferred by the second data. In another example, the second processed data may include information about the user's sleep stage, information about the user's stress index, or information about the user's sleep disorder inferred by the second data.

The third machine learning model 13 may use the third data collected by the third sensor 53 as input data to output the third processed data as output data. The third processed data may include, for example, information about an eye movement inferred by the third data. In another example, the third processed data may include information about the user's sleep stage, information about the user's stress index, or information about the user's sleep disorder inferred by the third data.

The fourth machine learning model 14 may use the fourth data collected by the fourth sensor 54 as input data to output the fourth processed data as output data. The fourth processed data may include, for example, information about an oxygen saturation level and/or an ECG inferred by the fourth data. In another example, the fourth processed data may include information about the user's sleep stage, information about the user's stress index, or information about the user's sleep disorder inferred by the fourth data.

The fifth machine learning model 15 may use the fifth data collected by the fifth sensor 55 as input data to output the fifth processed data as output data. The fifth processed data may include, for example, information about a movement inferred by the fifth data. In another example, the fifth processed data may include information about the user's sleep stage, information about the user's stress index, or information about the user's sleep disorder inferred by the fifth data.

In the disclosure, the hub device 1 may primarily process the data collected from the plurality of sensors 5 so that the data collected from the plurality of sensors 5 may be sent to the user device 2 in a communication scheme with low data transfer capacity.

The hub device 1 may process the data collected from the plurality of sensors 5, and transmit the processed data to the user device 2, in S3.

The hub device 1 may transmit the processed data to the user device 2 by wireless communication. In an embodiment, the communicator 130 of the hub device 1 may include a first communication module for receiving data collected from some (e.g., the fourth sensor 54) of the plurality of sensors 5 in a first wireless communication scheme, and a second communication module for transmitting the processed data to the user device 2 in a second wireless communication scheme.

The first wireless communication scheme and the second wireless communication scheme may be the same or different from each other.

In an embodiment of the disclosure, as the hub device 1 is equipped with both the first communication module for receiving data from some of the plurality of sensors 5 and the second communication module for communicating with the user device 2, the hub device 1 may be able to communicate with the plurality of sensors 5 and the user device 2 at the same time.

The user device 2 may process the processed data received from the hub device 1, in S4. For this, the user device 2 may be equipped with a machine learning model.

The processed data received from the hub device 1 may be processed by a machine learning model 21 installed in the user device 2.

The machine learning model 21 installed in the user device 2 may include an artificial neural network (deep neural network) model with several layers (e.g., an input layer, a hidden layer, and an output layer). The machine learning model 21 installed in the user device 2 may be configured in a perceptron structure that receives multiple signals and outputs one signal. The machine learning model 21 installed in the user device 2 may be trained for a purpose of estimating the user's sleep state based on the processed data processed by the hub device 1.

The machine learning model 21 installed in the user device 2 may use the processed data output by the machine learning model of the hub device 1 as input data to output sleep state information relating to the user's sleep state as output data.

The machine learning model 21 equipped in the user device 2 may use the first processed data, the second processed data, the third processed data, the fourth processed data and/or the fifth processed data as input data to output the sleep state information.

The sleep state information output by the machine learning model 21 installed in the user device 2 may include at least one of information about the user's sleep stage, information about the user's stress index, or information about the user's sleep disorder.

In the disclosure, as the data primarily output by the machine learning models 11, 12, 13, 14 and 15 installed in the hub device 1 is secondarily input to the machine learning model 21 installed in the user device 2 to output the sleep state information, the user's sleep state may be accurately estimated. In other words, in the disclosure, a large amount of data may be processed in stages, thereby accurately estimating the user's sleep state.

The user device 2 may transmit the sleep state information to the server device 3, in S5.

In the disclosure, instead of transmitting data directly involved with the user's privacy to the server device 3, only the sleep state information relating to the user's sleep state may be transmitted to the server device 3, thereby gaining the user's agreement on the data collection more easily.

The server device 3 may generate the control command to control the home appliance 4 based on the sleep state information, in S6 In this case, the home appliance 4 may include at least one home appliance 4 connected and registered with the user device 2. The server device 3 may store and/or manage the user account, and register the user device 2 and the home appliance 4 by associating them with the user account.

The at least one home appliance 4 connected and registered with the user device 2 may include the home appliance 4 registered with the user account with which the user device 2 is registered.

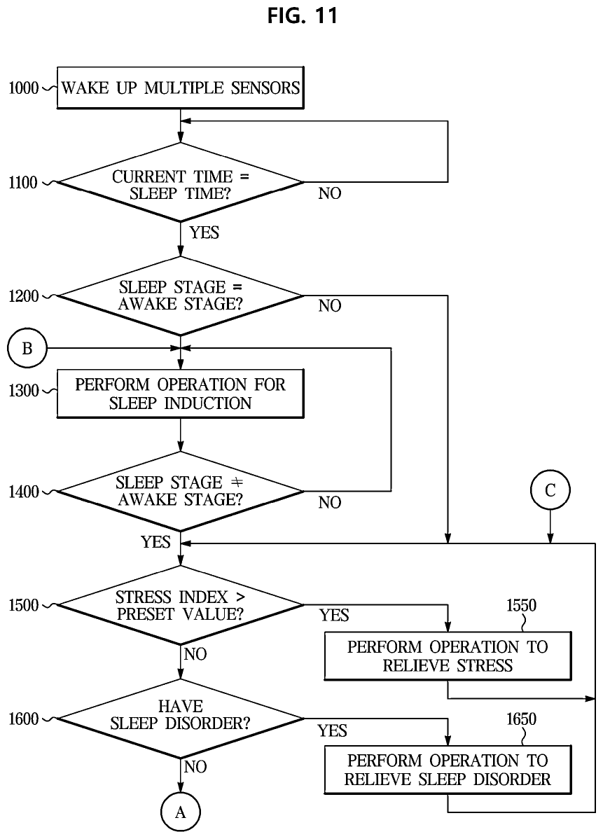

A procedure S6 for generating the control command to control the home appliance 4 based on the sleep state information will be described in detail later with reference to FIGS. 11 to 16. The server device 3 may transmit the control command to control the home appliance 4 to the home appliance 4, in S7.

In various embodiments, it is obvious that the procedure S6 for generating the control command to control the home appliance 4 based on the sleep state information and the procedure for transmitting the control command to control the home appliance 4 to the home appliance 4 may be performed by the user device 2 as well.

The home appliance 4 may perform a preset operation corresponding to the control command received from the server device 3, in S8.

The machine learning model stored in the user device 2 may be updated by an external server. For this, in various embodiments, data output by the machine learning models 11, 12, 13, 14 and 15 installed in the hub device 1 may be sent to the server device 3.

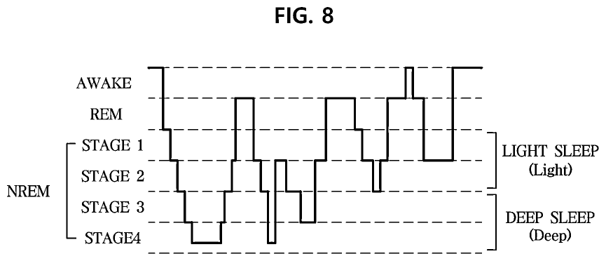

FIG. 8 is a diagram for describing sleep stages.

Sleep stages of humans may be classified into an awakening stage, a rapid eye movement (REM) sleep stage and a non REM (NREM) sleep stage.

The awakening stage corresponds to a stage in which a person is awake.

The REM sleep stage is a rapid eye movement sleep stage, which corresponds to a shallow sleep close to being awake, and is a stage of sleep distinguished by a rapid eye movement.

REM sleep of adults generally occurs at about 20 to 25% of the total amount of sleep. It repeats for about 90 to 120 minutes of nighttime sleep.

Brain's neural activity during the REM sleep is quite similar to when it is awake. However, the human body is in a relaxed state and thus becomes paralyzed. For this reason, the REM sleep stage is called paradoxical sleep. This means that brain waves are not suppressed during the REM sleep.

The NREM sleep stage is a non rapid eye movement sleep stage, and unlike the REM sleep, there is little eye movement in the NREM sleep stage.

One seldom dreams during the NREM sleep stage, and muscle movement is not suppressed as in REM sleep. People who do not properly go through the sleep stages may have sleep disorders (e.g., sleepwalking) because they are confined in NREM sleep and their muscles are not suppressed.

The NREM sleep stage may be divided into stage 1 (N1 stage), stage 2 (N2 stage), stage 3 (N3 stage) and stage 4 (N4 stage). In the NREM sleep stage, stage 1 (N1 stage) and stage 2 (N2 stage) may be classified as a light sleep stage, and stage 3 (N3 stage) and stage 4 (N4 stage) may be classified as a deep sleep stage.

In another example, the NREM sleep stage may be divided into stage 1 (N1 stage), stage 2 (N2 stage) and stage 3 (N3 stage).

In the NREM sleep stage, brainwave activity gradually slows down and the physiological function declines. During the NREM sleep stage, brain tissue cells and epithelial cells are regenerated, body energy is restored, and hormones for skeletal growth, protein synthesis and tissue regeneration are secreted.

The N1 stage is the most borderline phase in the sleep state, indicating a procedure in which human body slowly falls asleep. The N1 stage appears before falling into a deep sleep, and refers to a state of not falling into a deep sleep.

The N2 stage is a phase that goes into a deeper sleep from the borderline sleep phase. The brain wave pattern changes from slightly slow beta brain waves to slower theta brain waves, and eye movements stop and intermittent fast eye movements do not appear.

The N3 stage is an early stage of deep sleep, where the brainwave patterns change into delta brainwaves and muscle tension is relaxed so that there is little physical motion. In the N3 stage, snoring symptoms may appear.

The N4 stage corresponds to a deep sleep stage where it is very difficult to be awake. In the N4 stage, secretion of hormones for skeletal growth, protein synthesis and tissue regeneration may increase, and sleepwalking, bed-wetting, etc., may appear.

It is common for the first sleep cycle to begin when the person starts to fall asleep. In the first sleep cycle, the person enters into the NREM sleep stage from the awakening stage, goes through the N1, N2, N3 and N4 stages, and goes back to the N3, N2, N1, and REM sleep stages.

Subsequently, in the second sleep cycle, the person goes through the N1, N2, N3 and N4 stages and goes back to the N3 and N2 stages.

Subsequently, in the third sleep cycle, the person goes through the N3 stage and goes back to the N2, N1 and REM sleep stages.

Subsequently, in the fourth sleep cycle, the person enters back into the N1 and REM sleep stages after going through the N1 and N2 stages.

The person then naturally wakes up while going through the N1, N2 and REM sleep stages.

When the person wakes up during the N4 stage, he/she may become groggy as if he/she did not sleep even after getting out of bed. Hence, it may be desirable for the person to wake up during the N1 stage or REM sleep stage.

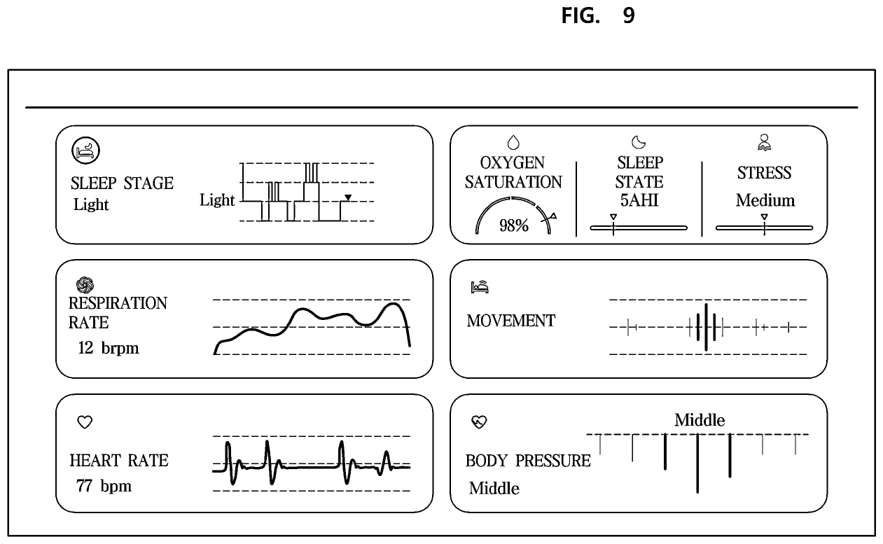

FIG. 9 illustrates an example of sleep state information derived by a sleep management system, according to an embodiment.

The sleep state information may be derived by the sleep management system in real time.

Referring to FIG. 9, the sleep state information may be derived by the sleep management system may include information about a sleep stage, information about oxygen saturation, information about sleep disorder, information about a stress index, information about a respiration rate, information about a movement, information about a heart rate and/or information about body pressure.

In various embodiment, the user device 2 (or the display device 41) may output the sleep state information through the output interface (e.g., a display). For example, based on obtaining of the sleep state information by the user device 2,
the information about a sleep stage may include information about a current sleep stage of the user and/or information about the user's sleep stages over time.

The information about the current sleep stage of the user may include information indicating which one of awakening stage, the REM sleep stage or the stage 1, stage 2 or stage 3 of NREM sleep the current sleep stage of the user corresponds to.

The information about the user's sleep stages over time may include information about changes in sleep stage from a time preset by the user with the user device 2, a time when the user lies or sits on the furniture 10 to fall asleep, a time when the user falls asleep and/or a time when the user runs the sleep management application to the current time.

When the information about the user's sleep stages over time is output by the user device 2 (or the display device 41), the information about the user's sleep stages over time may be represented in the form of a graph having the X-axis indicating time and the Y-axis indicating the user's sleep stages.

The information about oxygen saturation may include information about current oxygen saturation of the user and/or information about the user's oxygen saturation over time.

When the information about oxygen saturation is output by the user device 2 (or the display device 41), numerical values of the oxygen saturation may be output in percentage. Furthermore, when the information about oxygen saturation is output by the user device 2 (or the display device 41), whether the oxygen saturation of the user is normal according to medical standards may be displayed.

The information about sleep disorder may include information about a current sleep disorder of the user and/or information about the user's sleep disorders that appear over time.

The sleep disorders may include information about sleep disorder related to respiration such as apnea and hypopnea. The information about sleep disorder related to respiration such as apnea and hypopnea may include an apnea hypopnea index (AHI).

When the information about sleep disorders related to respiration is output by the user device 2 (or the display device 41), the AHI may be output with an indication whether the user's AHI is normal according to the medical standard.

The information about a stress index may include information about a stress level of the user. When the information about a stress index is output by the user device 2 (or the display device 41), the user's stress index may be output in a numerical value or in the form of a comparative word (e.g., high, medium, and low) with an indication whether the user's stress index is normal according to the medical standard.

The information about a respiration rate may include information about a current respiration rate of the user and/or information about the user's respiration rates over time.

When the information about the current respiration rate is output by the user device 2 (or the display device 41), the user's respiration rate may be output in a numerical value with an indication whether the user's respiration rate is normal according to the medical standard.

When the information about the user's respiration rates over time is output by the user device 2 (or the display device 41), the information about the user's respiration rates over time may be represented in the form of a graph having the X-axis indicating time and the Y-axis indicating the user's respiration rates.

The information about movement may include information about a degree of the user's movement and/or information about the user's movement degrees over time.

When the information about the current movement degree is output by the user device 2 (or the display device 41), the user's movement degree may be output in a numerical value or in the form of a comparative word (e.g., high, medium, and low).

When the information about movement degrees over time is output by the user device 2 (or the display device 41), the information about the user's movement degrees over time may be represented in the form of a graph having the X-axis indicating time and the Y-axis indicating the user's movement degrees.

The information about heart rate may include information about a current heart rate of the user and/or information about the user's heart rates over time.

When the information about the current heart rate is output by the user device 2 (or the display device 41), the user's heart rate may be output in a numerical value with an indication whether the user's heart rate is normal according to the medical standard.

When the information about the user's heart rates over time is output by the user device 2 (or the display device 41), the information about the user's heart rates over time may be represented in the form of a graph having the X-axis indicating time and the Y-axis indicating the user's heart rates.

The information about body pressure may include information about the user's body posture. The information about body posture may include information regarding whether the user's posture corresponds to lying on one's back, on the left side, on the right side, or on one's face, sitting up, etc.

When the information about body pressure is output by the user device 2 (or the display device 41), a term representing the user's posture may be used, and the form of a pressure distribution table based on the furniture 10 on which the user lies or sits may be output.

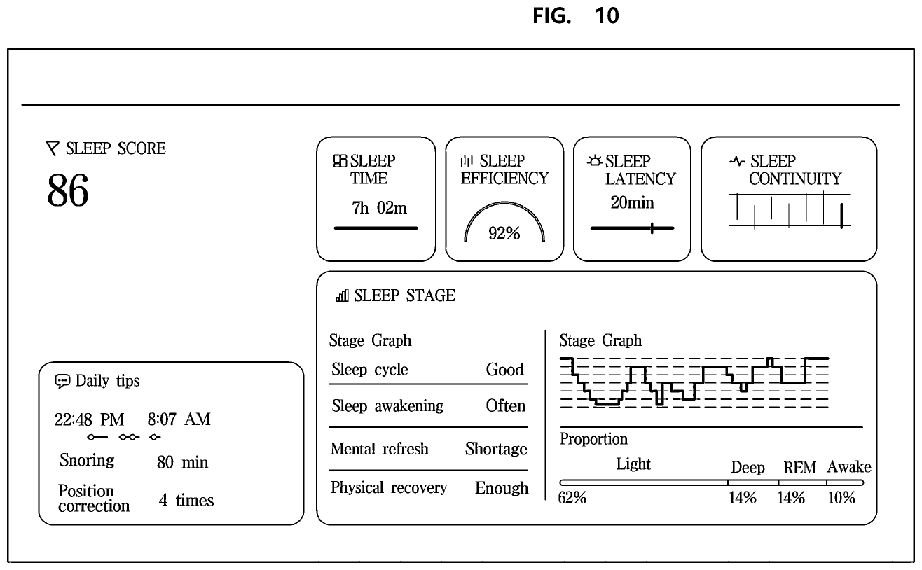

FIG. 10 illustrates an example of sleep summary information derived by a sleep management system, according to an embodiment.

The sleep summary information may include a collection of sleep state information obtained by the sleep management system in real time.

For example, the sleep summary information may include sleep score information, sleep time information, sleep efficiency information, sleep onset latency information, sleep continuity information, and/or sleep stage information corresponding to sleep quality.

The sleep score corresponding to sleep quality may be determined based on the sleep state information obtained in real time during the user's sleep. For example, the user device 2 and/or the server device 3 may calculate the sleep score based on the sleep state information obtained in real time during the user's sleep. Information about the sleep score may be calculated based on the sleep stage, oxygen saturation, sleep disorder, stress index, respiration rate, movement, heart rate and/or body pressure during the user's sleep.

The sleep time information may include information about a time from when the user goes through a change from the awakening state to a sleep state to when the user finally wakes up.

In another example, the sleep time information may include information about a time from when the person lies on the furniture 10 to when he/she finally wakes up.

The information about sleep efficiency may be determined based on a time proportion of a preset stage (e.g., the NREM sleep stage) in the sleep stage. The user device 2 and/or the server device 3 may calculate sleep efficiency based on the sleep state information obtained in real time during the user's sleep. In an embodiment, the user device 2 and/or the server device 3 may determine sleep efficiency based on a time proportion of a preset stage (e.g., the NREM sleep stage) in the user's sleep stage.

The information about the sleep onset latency (SOL) may include information about a time required for the sleep stage to be changed from the awakening stage to the NREM sleep stage.

The information about the sleep continuity may be determined based on a time for which the sleep phase does not change into the awakening phase (and/or the REM sleep stage) but the NREM sleep stage is maintained. The user device 2 and/or the server device 3 may determine sleep continuity based on the sleep state information obtained in real time during the user's sleep. In an embodiment, the user device 2 and/or the server device 3 may determine sleep continuity based on a time for which a preset stage (e.g., the NREM sleep stage) among the user's sleep stage is maintained.

The information about the sleep stage may include information about changes in sleep stage over time. The information about the sleep stage may include information about a time proportion of each sleep stage during the user's sleep, information about whether the user's sleep cycle corresponds to a normal range, and information about an extent to which the body is repaired, an extent to which the brain is repaired and/or an extent of periodic awakening determined based on a time proportion of each sleep phase during the user's sleep.

The sleep summary information is not limited to the above example, and may include various information relating to the user's sleep. For example, the sleep summary information may include information about the user's snoring time and information about the number of changes in the user's posture.

The sleep state information and the sleep summary information as described with reference to FIGS. 9 and 10 may be provided for the user by the user device 2 or by the home appliance 4 (e.g., the display device 41, the speaker 46, etc.).

For example, the user may use the user device 2 to run a sleep management application, and check the sleep state information and the sleep summary information through an interface provided by the sleep management application.

In another example, the server device 3 may control the home appliance 4 (e.g., the display device 41, the speaker 46, etc.) to provide the sleep summary information when the user wakes up from the sleep.

In various embodiments, the user may share the sleep state information and/or sleep summary information with other users through the user device 2. For example, the user may run the sleep management application, and transmit the sleep state information and/or the sleep summary information to the other users' devices through the interface provided by the sleep management application.

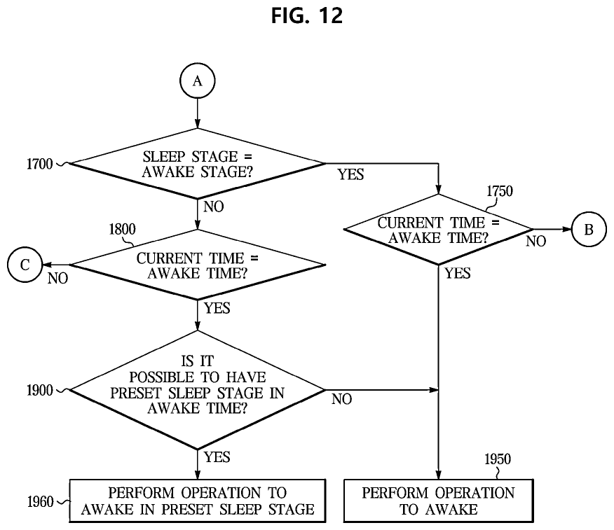

FIGS. 11 and 12 illustrate an example of a flowchart of a sleep management method, according to an embodiment.

As described above with reference to FIG. 6, in an embodiment, the server device 3 may control the home appliance 4 based on the sleep state information.

Referring to FIGS. 11 and 12, the hub device 1 may wake up the plurality of sensors 5 based on a preset condition being satisfied.

For example, the hub device 1 may wake up the plurality of sensors 5 based on determining that there is a user on the furniture 10, in 1000.

In another example, the user device 2 may transmit a request to start sleep management to the hub device 1 based on receiving of a user input corresponding to start of sleep, and the hub device 1 may wake up the plurality of sensors 5 based on receiving of the request to start sleep management through the user device 2.

In another example, the user device 2 may receive information about an expected sleep time from the user and forward the information to the hub device 1. The hub device 1 may wake up the plurality of sensors 5 based on reaching the expected sleep time.

The server device 3 may determine whether the current time corresponds to a sleeping time, in 1100.

For example, the user may preset the sleeping time through the user device 2. The sleeping time may be set to a time range or a time. For example, the sleeping time may be set by the user to “10 p.m. to 11 p.m., or set to “10 p.m.”.

The server device 3 may determine that the current time corresponds to the sleeping time when the current time corresponds to the sleeping time set by the user.

In another example, when the user inputs a command corresponding to start of sleep through the user device 2, the server device 3 may determine that the current time corresponds to the sleeping time.

The command corresponding to start of sleep may be received through the user interface 240 of the user device 2. For example, the command corresponding to start of sleep may be received by the user device 2 through a touch input and/or sound input.

In various embodiments, when the user inputs an automatic sleep management request through the user device 2, the server device 3 may determine that the current time corresponds to the sleeping time based on a preset condition being satisfied. In an embodiment, the server device 3 may determine whether the current time corresponds to the sleeping time based on the resultant information. For example, the server device 3 may determine that the current time corresponds to the sleeping time when the user's preset posture (e.g., posture of lying on the back or on the face) is maintained for a preset period of time.

When the current time corresponds to the sleeping time in 1100, the server device 3 may control the home appliance 4 based on the sleep state information received from the user device 2.

In an embodiment, when the current time corresponds to the sleeping time in 1100 and the user's sleep stage corresponds to a preset stage (e.g., awakening stage) in 1200, the server device 3 may control the home appliance 4 to perform a preset operation to induce sleep in 1300.

For example, the user device 2 may transmit a request for start of sleep to the server device 3 based on receiving of a command corresponding to start of sleep from the user, and the server device 3 may receive the request for the start of sleep, and control the home appliance 4 to perform a preset operation to induce sleep when the user's sleep stage corresponds to a preset stage (e.g., awakening stage).

The preset operation to induce sleep may include a preset operation to be performed by each of the at least one home appliance 4.

The preset operation to induce sleep may be set in advance, and may be changeable by the user through the user device 2.

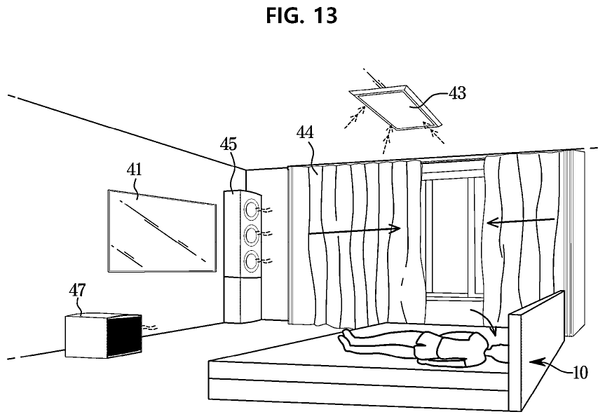

FIG. 13 illustrates an example of a preset operation to induce sleep of the user.

Referring to FIG. 13, the server device 3 may control the display device 41 to perform the preset operation to induce sleep.

For example, the server device 3 may control the display device 41 so that the brightness of an image output from the display device 41 gradually becomes darker. In another example, the display device 41 may be controlled to play preset music (e.g., music to induce sleep).

In various embodiments, the server device 3 may control the speaker 46 to play certain music (e.g., music to induce sleep).

The server device 3 may control the lighting device 43 to perform a preset operation to induce sleep. For example, the server device 3 may control the lighting device 43 so that the brightness of light output from the lighting device 43 gradually becomes darker. In another example, the server device 3 may control the lighting device 43 so that color of the light output from the lighting device 43 is changed to a preset color (e.g., a color having a color temperature of light of 2000K or less).

The server device 3 may control the automatic curtain open/close device 44 to perform a preset operation to induce sleep. For example, the server device 3 may control the automatic curtain open/close device 44 to close the curtain.

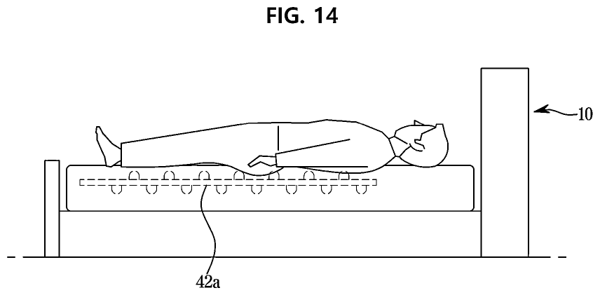

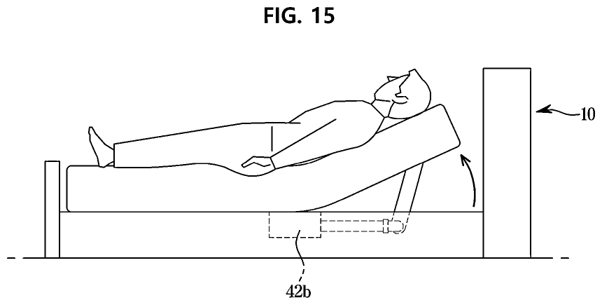

The furniture control device 42 may include a vibration element 42a that is able to transmit vibration to the user who lies or sits on the furniture 10 and/or an actuator 42b that is able to change the posture of the user by changing the structure of the furniture 10 (see FIGS. 14 and 15).

The server device 3 may control the furniture control device 42 to perform a preset operation to induce sleep.

For example, the server device 3 may control the vibration element 42a to output vibration at preset intervals to ease the tension of the user.

In another example, the server device 3 may control the actuator 42b to change the structure of the furniture so that the user's posture is changed to a preset posture (lying on his/her back) that makes it easy to fall sleep.

The server device 3 may control the air conditioner 45 and/or the air purifier 47 to perform a preset operation to induce sleep. For example, the server device 3 may control the air conditioner and/or the air purifier 47 to operate in a sleep mode.

While operating in the sleep mode, the air purifier 45 and/or the air purifier 47 may minimize noise, for example, by controlling the fan speed to slow down.

The preset operation to induce sleep that may be performed by the home appliance 4 is not limited to the above example, and may be obviously changed according to the user's setting and the type of the home appliance 4.

Turning back to FIG. 11, the server device 3 may determine whether the user's sleep stage corresponds to the awakening stage, in 1400.

When the user's sleep stage does not correspond to the awakening stage in 1400, the server device 3 may control the home appliance 4 to perform a preset operation to reduce the user's stress index or relieve the sleep disorder.

In an embodiment, when the user's sleep stage corresponds to the REM sleep stage or NREM sleep stage, the server device 3 may control the home appliance 4 to perform a preset operation to reduce the user's stress index or relieve the sleep disorder.

When the user's sleep stage corresponds to the NREM sleep stage, the server device 3 may control the home appliance 4 to perform a preset operation to reduce the user's stress index or relieve the sleep disorder.

According to an embodiment of the disclosure, by controlling the home appliance 4 to perform a preset operation to reduce the user's stress index or relieve the sleep disorder only when the user's sleep stage corresponds to the NREM sleep stage, the user may be prevented from waking up during the REM sleep stage due to an operation of the home appliance 4.

The preset operation to reduce stress that may be performed by the home appliance 4 is not limited to the above example, and may be obviously changed according to the user's setting and the type of the home appliance 4.

The server device 3 may control the home appliance 4 in 1550 to perform the preset operation to reduce the user's stress in response to the user's stress index exceeding a preset value in 1500.

FIG. 14 illustrates an example of a preset operation to relieve stress of the user.

Referring to FIG. 14, the server device 3 may control the lighting device 43 to perform the preset operation to reduce the user's stress.

For example, the server device 3 may control the lighting device 43 to output light of a preset color (e.g., a color having a color temperature of light of 1000K or less).

The server device 3 may control the air conditioner 45 to perform the preset operation to reduce the user's stress.

For example, the server device 3 may control the air conditioner 45 to slightly increase the fan speed so that the wind output from the air conditioner 45 may reach the user.

The server device 3 may control the furniture control device 42 to perform a preset operation to reduce the user's stress. The furniture control device 42 may include the vibration element 42a that is able to transmit vibration to the user who lies or sits on the furniture 10.

For example, the server device 3 may control the vibration element 42a to output vibration corresponding to the user's heart rate.

In an embodiment of the disclosure, by reducing the user's stress index during the sleep, quality sleep of the user may be induced.

Turning back to FIG. 11, in response to the appearance of a preset sleep disorder to the user in 1600, the server device 3 may control the home appliance 4 to perform a preset operation to relieve the sleep disorder that appears, in 1650.

The preset operation to relieve the sleep disorder that may be performed by the home appliance 4 is not limited to the above example, and may be obviously changed according to the user's setting and the type of the home appliance 4.

FIG. 15 illustrates an example of a preset operation to relieve sleep disorders of the user.

Referring to FIG. 15, the server device 3 may control the air conditioner 45 to perform a preset operation to relieve the sleep disorder.

For example, the server device 3 may turn off the air conditioner 45.

The server device 3 may control the furniture control device 42 to perform a preset operation to relieve the sleep disorder. The furniture control device 42 may include the actuator 42b that is able to change the user's posture by changing the structure of the furniture 10.

For example, the server device 3 may control the actuator 42b to change the structure of the furniture so that the user's posture is changed to a preset posture (in which the user's head is located higher than the torso and leg portions) that eases the user's breathing.

In an embodiment of the disclosure, by relieving the sleep disorder that appears during the sleep, quality sleep of the user may be induced.

Turning back to FIG. 12, the server device 3 may control the home appliance 4 to perform a preset operation for awakening in 1950, based on the sleep stage not corresponding to the awakening stage in 1700 and the current time corresponding to awakening time in 1800.

For example, the user may preset the awakening time through the user device 2. The awakening time may be set to a time range or a time. For example, the awakening time may be set by the user to “7 a.m. to 8 a.m., or set to “7 a.m.”.

The server device 3 may determine that the current time corresponds to the awakening time when the current time corresponds to the awakening time set by the user.

In another example, when the user inputs a command corresponding to start of awakening through the user device 2, the server device 3 may determine that the current time corresponds to the awakening time.

The command corresponding to the start of awakening may be received through the user interface 240 of the user device 2. For example, the command corresponding to the start of awakening may be received by the user device 2 through a touch input and/or sound input.

In various embodiments, when the user inputs an automatic sleep management request through the user device 2, the server device 3 may determine that the current time corresponds to the awakening time based on a preset condition being satisfied. In an embodiment, the server device 3 may determine whether the current time corresponds to the awakening time based on the resultant information. For example, the server device 3 may determine that the current time corresponds to the awakening time when the user's sleep stage is maintained for a preset period of time (e.g., 10 minutes) as the awakening stage.

As described above, it may be desirable for the person to wake up during the N1 stage or REM sleep stage.

The server device 3 may determine a chance that a preset sleep stage (e.g., the REM sleep stage and/or the NREM sleep stage) enters the N1 stage within the awakening time based on the sleep state information, in 1900.

In a case that the awakening time is set to a time range, the determining of the chance of entering a preset sleep stage (e.g., the REM sleep stage and/or the N1 stage of the NREM sleep stage) within the awakening time may include determining whether the user's sleep stage is going to enter the preset sleep stage (e.g., the REM sleep stage and/or the N1 stage of the NREM sleep stage) within the set time range.

In a case that the awakening time is set to a time, the determining of the chance of entering a preset sleep stage (e.g., the REM sleep stage and/or the N1 stage of the NREM sleep stage) may include determining whether the user's sleep stage is going to enter the preset sleep stage (e.g., the REM sleep stage and/or the N1 stage of the NREM sleep stage) within a preset time (e.g., 15 minutes) around the time corresponding to the awakening time.

When there is a chance that the user's sleep stage is going to enter the preset stage (e.g., the REM sleep stage and/or the N1 stage of the NREM sleep stage) within the awakening time in 1900, the server device 3 may control the home appliance 4 to perform a preset operation to wake up the user based on the user's sleep stage entering the preset stage (e.g., the REM sleep stage and/or the N1 stage of the NREM sleep stage) in 1960.

In other words, the server device 3 may control the home appliance 4 to perform the preset operation to wake up the user when the current time corresponds to the awakening time and the user's sleep stage corresponds to the preset stage (e.g., the REM sleep stage and/or the N1 stage of the NREM sleep stage).

In the disclosure, by waking up the user during the REM sleep stage and/or the N1 stage of the NREM sleep stage, the user may be led to feel fresh when he/she is awaken.

On the other hand, when there is no chance that the user's sleep stage is going to enter the preset stage (e.g., the REM sleep stage and/or the N1 stage of the NREM sleep stage) within the awakening time in 1900, and when the current time corresponds to the awakening time, the server device 3 may control the home appliance 4 to perform a preset operation to wake up the user in 1950.

In an embodiment, the server device 3 may control the home appliance 4 to perform a preset operation for waking up the user in 1950, when the user's sleep stage corresponds to the awakening stage and the current time corresponds to the awakening time in 1750.

In an embodiment, the server device 3 may perform the operation for awakening or perform an operation to induce sleep depending on the user input, when the user's sleep stage corresponds to the awakening stage and the current time does not correspond to the awakening time in 1750.

For example, when the current time in the user's sleep stage does not correspond to the awakening time, the server device 3 may perform the operation for awakening in 1950, based on the user device 2 receiving a user input to request the start of awakening.

In another example, when the current time in the user's sleep stage does not correspond to the awakening time, the server device 3 may perform the operation to induce sleep, based on the user device 2 failing to receive the user input to request the start of awakening within a preset time, in 1300.

In another example, when the current time in the user's sleep stage does not correspond to the awakening time, the server device 3 may perform the operation to induce sleep based on the user device 2 receiving a user input to request to start to sleep again in 1300.

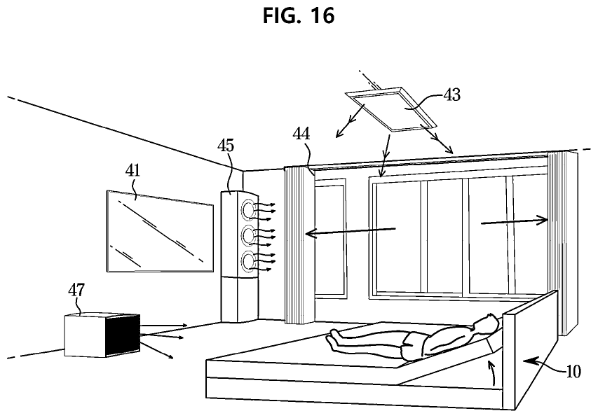

FIG. 16 illustrates an example of a preset operation to induce wakeup of the user.

Referring to FIG. 16, the server device 3 may control the display device 41 to perform the preset operation to induce sleep.

For example, the server device 3 may control the display device 41 so that the brightness of an image output from the display device 41 gradually becomes brighter. In another example, the display device 41 may be controlled to play preset music (e.g., music to induce wakeup).

In various embodiments, the server device 3 may control the speaker 46 to play preset music (e.g., music to induce wakeup).

The server device 3 may control the lighting device 43 to perform a preset operation to induce wakeup. For example, the server device 3 may control the lighting device 43 so that the brightness of light output from the lighting device 43 gradually becomes brighter. In another example, the server device 3 may control the lighting device 43 to output light similar to natural light.

The server device 3 may control the automatic curtain open/close device 44 to perform a preset operation to induce wakeup. For example, the server device 3 may control the automatic curtain open/close device 44 to open the curtain.

The server device 3 may control the furniture control device 42 to perform a preset operation to induce wakeup. The furniture control device 42 may include the vibration element 42a that is able to transmit vibration to the user who lies or sits on the furniture 10 and/or the actuator 42b that is able to change the posture of the user by changing the structure of the furniture 10.

For example, the server device 3 may control the vibration element 42a to output vibration at preset intervals corresponding to the user's heart rate.

In another example, the server device 3 may control the actuator 42b to change the structure of the furniture so that the user's posture is changed to a preset posture (in which the upper body is located higher than the lower body) that makes it easy for the user to wake up.

The server device 3 may control the air conditioner 45 and/or the air purifier 47 to perform a preset operation to induce wakeup. For example, the server device 3 may control the air conditioner and/or the air purifier 47 to operate in an awakening mode.

While operating in the awakening mode, the air purifier 45 and/or the air purifier 47 may control the fan at high speed, giving pleasant feeling to the user.

The preset operation to induce wakeup that may be performed by the home appliance 4 is not limited to the above example, and may be obviously changed according to the user's setting and the type of the home appliance 4.

In various embodiments, the operations as described above with reference to FIGS. 11 to 16 may be performed by the user device 2 as well. For example, the user device 2 may control the home appliance 4 based on the sleep state information.

According to an embodiment of the disclosure, the user's quality sleep may be induced.

According to an embodiment of the disclosure, a sleep management system may include the plurality of sensors 5 including the first sensor 51, 52, 53, 54 or 55 and the second sensor 51, 52, 53, 54 or 55 for collecting data of a user; the hub device 1 configured to receive first data collected by the first sensor 51, 52, 53, 54 or 55 and second data collected by the second sensor 51, 52, 53, 54 or 55, obtain first processed data based on processing of the first data, and obtain second processed data based on processing of the second data; the user device 2 configured to receive the first processed data and the second processed data from the hub device 1 through wireless communication, and obtain sleep state information relating to the user's sleep state by inputting the first processed data and the second processed data to a machine learning model; and the server device 3 configured to receive the sleep state information from the user device 2 by wireless communication.

The hub device 1 may obtain the first processed data by inputting the first data to a first machine learning model 11, 12, 13, 14 or 15 and obtain the second processed data by inputting the second data to a second machine learning model 11, 12, 13, 14 or 15.

The hub device 1 may receive data of the user from the plurality of sensors 5 by wired communication.

The hub device 1 may receive the first data from the first sensor 51, 52 or 53 by wired communication and receive the second data from the second sensor 54 or 55 by wireless communication.

The plurality of sensors 5 may include at least two of a pressure sensor, a UWB sensor, an oxygen saturation sensor, an ECG sensor, or an acceleration sensor.

The sleep state information relating to the user's sleep state may include at least one of information about the user's sleep stage, information about the user's stress index, or information about the user's sleep disorder.

The user device 2 may store a sleep management application including the machine learning model 21.

The sleep management application may be downloadable from an external server.

The server device 3 may control the at least one home appliance connected and registered with the user device 2 based on the sleep state information.

The server device 3 may control the at least one home appliance 4 to perform a preset operation to wake up the user in response to a current time corresponding to an awakening time and the user's sleep stage corresponding to a preset stage.

The server device 3 may control the at least one home appliance 4 to perform a preset operation to induce sleep of the user in response to a current time corresponding to a sleeping time and the user's sleep stage corresponding to a preset stage.

The server device 3 may control the at least one home appliance 4 to perform a preset operation to relieve the user's stress in response to the user's stress index exceeding a preset value.

The server device 3 may control the at least one home appliance 4 to perform a preset operation to relieve preset sleep disorder in response to the preset sleep disorder appearing to the user.

The hub device 1 may include a printed circuit board wiredly connected to the plurality of sensors 5.

According to an embodiment of the disclosure, a sleep management method may include receiving, by the hub device 1, first data collected by the first sensor 51, 52, 53, 54 or 55 and second data collected by the second sensor 51, 52, 53, 54 or 55; obtaining, by the hub device 1, first processed data based on processing of the first data and second processed data based on processing of the second data; transmitting, by the hub device 1, the first processed data and the second processed data to the user device 2 through wireless communication; obtaining, by the user device 2, sleep state information relating to a sleep state of the user by inputting the first processed data and the second processed data received from the hub device 1 to a machine learning model through wireless communication; and transmitting, by the user device 2, the sleep state information to the server device 3 by wireless communication.

The obtaining of the first processed data and the second processed data by the hub device 1 may include obtaining the first processed data by inputting the first data to a first machine learning model 11, 12, 13, 14 or 15 and obtaining the second processed data by inputting the second data to a second machine learning model 11, 12, 13, 14 or 15.

The receiving of the first data and the second data by the hub device 1 may include receiving the first data from the first sensor 51, 52 or 53 by wired communication and receiving the second data from the second sensor 51, 52 or 53 by wired communication.

The receiving of the first data and the second data by the hub device 1 may include receiving the first data from the first sensor 51, 52 or 53 by wired communication and receiving the second data from the second sensor 54 or 55 by wireless communication.

The sleep management method may further include controlling, by the server device 3, the at least one home appliance connected and registered with the user device 2 based on the sleep state information.

The controlling of the at least one home appliance 4 by the server device 3 may include controlling the at least one home appliance 4 to perform a first preset operation to wake up the user in response to a current time corresponding to an awakening time and the user's sleep stage corresponding to a first preset stage; controlling the at least one home appliance 4 to perform a second preset operation to induce sleep of the user in response to a current time corresponding to a sleeping time and the user's sleep stage corresponding to a second preset stage; controlling the at least one home appliance 4 to perform a third preset operation to relieve the user's stress in response to the user's stress index exceeding a preset value; or controlling the at least one home appliance 4 to perform a fourth preset operation to relieve preset sleep disorder in response to the preset sleep disorder appearing to the user.

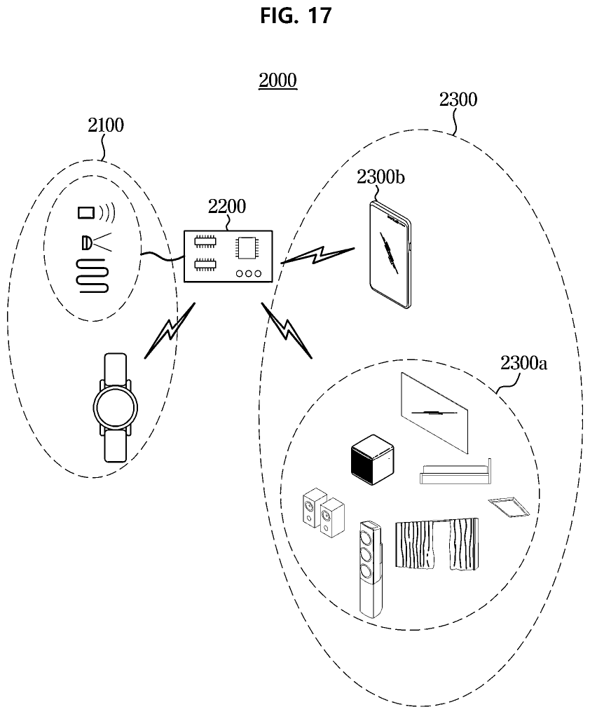

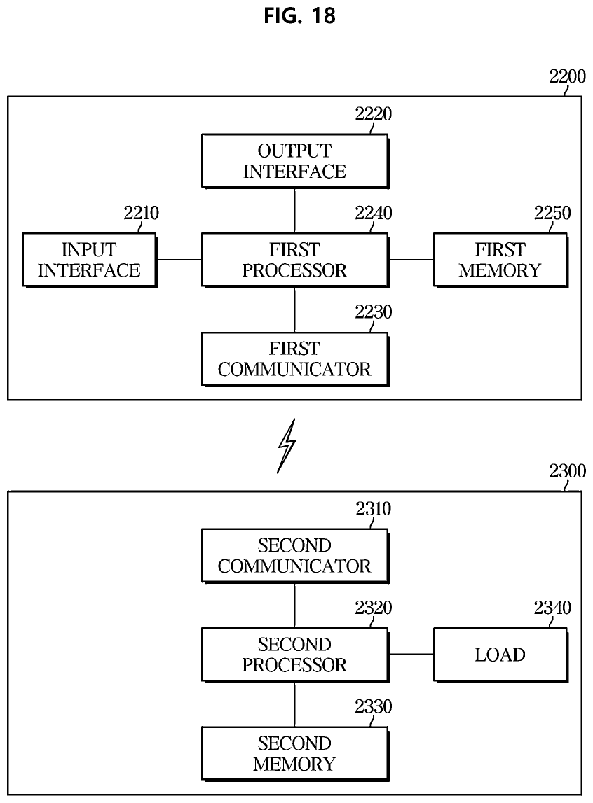

FIG. 17 is a configuration diagram of a sleep management system according to an embodiment, which will be described with reference to FIGS. 18 to 21.

A sleep management system 2000 may include a sensor 2100, a hub device 2200, and electronic devices 2300.

There may be one or more sensors 2100.

The sensor 2100 is able to communicate with the hub device. In other words, the sensor 2100 is able to communicate with the electronic device. The sensor 2100 may include a communicator for communicating with the hub device.

The sensor 2100 may detect a biometric signal of the user and transmit the detected biometric signal to the hub device 2200.

The sensor 2100 may be arranged at home and in a room where the user goes to sleep.

The sensor 2100 may be arranged in at least one electronic device, in the hub device or at a bed.

The sensor 2100 may be a contact type or non-contact type sensor.

The sensor 2100 may include at least one of an image sensor for obtaining an image of the user and a microphone for receiving the sound of the user's breathing.

The image sensor may include a CCD or CMOS image sensor.

The image sensor may include a camera. The image sensor may include a 3D spatial awareness camera such as a time of flight (ToF) camera, a stereo camera, etc.

The image sensor and the microphone may be arranged in a room or bed where the user goes to sleep.

The sensor 2100 may include at least one of a pressure sensor for detecting pressure applied by the user, a temperature sensor for detecting the user's body temperature, a heart rate sensor for detecting the user's heart rate, an ECG sensor for detecting the user's ECG, an oxygen saturation sensor for detecting the user's oxygen saturation, an acceleration sensor and a radar sensor for detecting the user's movement.

The pressure sensor may include a single strip force sensitive resistor (FSR).

The single strip FSR is a sensor for detecting pressure, weight, touch or the like based on a principle in which a resistance value decreases when pressure is applied.

The pressure sensor may also include a liquid metal based pressure sensor.

The pressure sensor may also include a piezo pressure sensor.

The pressure sensor may be arranged on a bed in the room where the user goes to sleep.

The radar sensor may include a transmit antenna (or a transmit antenna array) for emitting transmission radio waves toward a sleep area in the room, and a receive antenna (or a receive antenna array) for receiving reflected radio waves reflecting from an obstacle or the user in the sleep area.

The radar sensor may obtain radar information from the transmission radio waves transmitted by the transmit antenna and reflected radio waves received by the receive antenna.

The radar sensor may include at least one of an ultra-wideband (UWB) radar sensor for detecting presence and movement of the user by using high frequency radio waves, and a millimeter wave (mmWave) radar sensor for detecting the presence and movement of the user by using millimeter radio waves.

The radar sensor may be arranged in the room or bed where the user goes to sleep.

The image sensor, the radar sensor and the microphone may be non-contact type sensors.

The pressure sensor, the temperature sensor, the ECG sensor, the acceleration sensor and the heart rate sensor may be contact type sensors.

The sensor 2100 may also be arranged in a wearable device. For example, the sensor arranged in the wearable device may include the heart rate sensor, a bioelectrical impedance analysis sensor, a body temperature sensor and the acceleration sensor.

The hub device 2200 determines the user's sleep state based on a biometric signal detected by the one or more sensors 2100.

The determining of the user's sleep state may include determining whether the user is in a pre-sleep state, a sleeping state, or a post-sleep state.

The pre-sleep state may be a state when the user is at a sleep preparation stage. The sleeping state may be a state when the user is at a sleeping stage. The post-sleep state may be a state when the user is at an awake stage.

The sleeping stage may include a non-REM sleep stage and a REM sleep stage.

The hub device 2200 may determine whether the user is at the sleep preparation stage, the sleeping stage or the awake stage. In other words, the hub device 2200 may determine the user's current stage related to sleep.

The hub device 2200 may generate a control command to control an operation of one or more electronic devices 2300 (2300a and 2300b) based on the determined current stage of the user, and transmit the control command to the one or more electronic devices 2300.

The hub device 2200 may also transmit stage information corresponding to the user's current stage related to sleep to the one or more electronic device 2300.

Furthermore, the hub device 2200 may directly controls operations of the one or more electronic devices 2300.

The hub device 2200 may store control information for the one or more electronic devices 2300 for each user stage, and control the operation of the one or more electronic devices 2300 at each stage based on the user's identity information and the control information for the one or more electronic devices 2300.

The hub device 2200 may also determine priorities of a plurality of users when it is determined that there are the plurality of users in the same room, determine identity information of a user having the highest of the determined priorities, obtain control information for the one or more electronic devices 2300 for each stage corresponding to the identity information of the user, and control the operation for the one or more electronic devices 2300 based on the obtained control information for the one or more electronic devices 2300 for each stage.

The hub device 2200 may also communicate with the electronic devices 2300 arranged at home and obtain identification information and location information of the electronic devices 2300 through the communication with the electronic devices 2300.

The hub device 2200 may obtain identification information of the room where each electronic device 2300 is located, based on the location information of the electronic devices 2300.

The hub device 2200 may store the identification information of the electronic device 2300 arranged in each room by matching it with the identification information of the room.

The hub device 2200 may also receive, from the user device 2300b, identification information of the electronic device 2300 in each room and identity information of a user in each room, and store the received identification information of the electronic device 2300 in each room and identity information of the user in each room.

The hub device 2200 may receive control information for the electronic devices 2300 for each stage of each user from the user device 2300b and store the control information for the electronic devices 2300 for each stage of each user.

The hub device 2200 may also receive priority information of the plurality of users from the user device 2300b and store the received priority information of the plurality of users.

The electronic device 2300 may be a device that is capable of communicating with the hub device 2200.

The electronic device 2300 may also communicate with other electronic devices.

The electronic device 2300 may include one or more home appliances 2300a arranged at home. For example, the home appliance 2300a may include at least one of an automatic curtain open/close device, a TV, a projector, a soundbar, a lighting device, an air conditioner and a humidifier.

The electronic device 2300 may be a multimedia device or a display device.

The electronic device 2300 may include the user device 2300b such as a personal computer, a terminal, a mobile phone, a smart phone, a handheld device, and a wearable device.

The electronic device 2300 controls operation based on the control command received from the hub device 2200.

The electronic device 2300 may control one or more functions in controlling the operation.

When receiving stage information corresponding to the user's sleep state from the hub device 2200, the electronic device 2300 may control one or more functions based on the received stage information. In this case, the electronic device 2300 may store control information for each stage. The control information for each stage may be configured by the user.

FIG. 18 is a control block diagram of a hub device and electronic devices arranged in a sleep management system, according to an embodiment.

At least one component may be added to or omitted from the hub device 2200 to correspond to the performance of the hub device 2200 as shown in FIG. 18. Furthermore, it will be obvious to those of ordinary skill in the art that the relative positions of the components may be changed to correspond to the performance or structure of the hub device 2200.

At least one component may be added to or omitted from the electronic device 2300 to correspond to the performance of the electronic device 2300 as shown in FIG. 18. Furthermore, it will be obvious to those of ordinary skill in the art that the relative positions of the components may be changed to correspond to the performance or structure of the electronic device 2300.

To distinguish between components of the hub device 2200 and the electronic device 2300 having the same name, the component of the hub device 2200 is expressed with a modifier ‘first’ and the component of the electronic device 2300 is expressed with a modifier ‘second’.

The hub device 2200 includes an input interface 2210, an output interface 2220, a first communicator 2230, a first processor 2240 and a first memory 2250.

The input interface 2210 receives a user input and forwards the received user input to the first processor 2240.

The user input may include sleep management mode on information, sleep management mode off information, and awake time information corresponding to an awake time range.

The user input may include control information for each electronic device.

The control information for the electronic device 2300 may include control information corresponding to each of the sleep preparation stage, the sleeping stage and the awake stage.

The control information for each electronic device 2300 may include on information and off information for the electronic device, and further include target control value information.

When the electronic device 2300 is a lighting device, the control information for the electronic device 2300 may include off information for the sleep preparation stage, off information for the sleeping stage, and on information for the awake stage.

When the electronic device 2300 is the lighting device, the control information for the electronic device may include room light intensity information for the sleep preparation stage, room light intensity information for the sleeping stage, and light intensity information for the awake stage.

When the electronic device 2300 is an air conditioner, the control information for the electronic device may include on information for the sleep preparation stage, on information for the sleeping stage, and off information for the awake stage.

When the electronic device 2300 is the air conditioner, the control information for the electronic device may include room temperature information, airflow information and wind direction information for the sleep preparation stage, room temperature information, airflow information and wind direction information for the sleeping stage, room temperature information, airflow information and wind direction information for the awake stage.

When the electronic device 2300 is a humidifier, the control information for the electronic device may include on information for the sleep preparation stage, on information for the sleeping stage, and off information for the awake stage.

When the electronic device 2300 is the humidifier, the control information for the electronic device may include room humidity information for the sleep preparation stage, room humidity information for the sleeping stage, and humidity information for the awake stage.

The user input may include identification information for each room, identity information for each user and identification information for each electronic device.

The input interface 2210 may receive room identification information and user identification information that are matched with room identification information for each room.

When there are multiple users, the input interface 2210 may further receive priority information of the users.

The input interface 2210 may include hardware devices such as many different buttons or switches, a pedal, a keyboard, a mouse, a track ball, various levers, a handle, a stick, or the like.

Alternatively, the input interface 2210 may include a graphical user interface (GUI) such as a touch pad, i.e., a software device. The touch pad may be implemented with a touch screen panel (TSP), thus forming an interlayer structure with a screen.

The output interface 2220 may output information corresponding to a user input received at the input interface 2210.

The output interface 2220 may output information corresponding to a control command of the first processor 2240.

The output interface 2220 may include at least one of a display for displaying images and a speaker for outputting sound.

The display may include a cathode ray tube (CRT), a digital light processing (DLP) panel, a plasma display panel (PDP), a liquid crystal display (LCD) panel, an electro luminescence (EL) panel, an electrophoretic display (EPD) panel, an electrochromic display (ECD) panel, a light emitting diode (LED) panel, an organic light emitting diode (OLED) panel, etc., but is not limited thereto.

The display may be implemented with the TSP that forms the interlayer structure with the touch pad.

The speaker may control audio volume in response to a control command of the first processor 2240.

The first communicator 2230 may include one or more components that enable communication between components in the hub device 2200.

The first communicator 2230 may include one or more components that enable communication with an external device, for example, at least one of a short-range communication module, a wired communication module, and a wireless communication module. The external device may include a plurality of sensors, a home appliance, a server device and a user device.

The first communicator 2230 may include both wired and wireless networks.

The first processor 2240 controls general operation of the hub device 2200.

There may be one or more first processors 2240. In other words, there may be at least one first processor 2240.

The first processor 2240 uses data stored in the first memory 2250 to perform control related to sleep management. It is also possible that the first processor 2240 uses data stored in the first memory 2250 to perform control related to operation of the hub device 2200.

The first processor 2240 may control activation of the plurality of sensors when receiving on information for a sleep management mode from the input interface 2210.

The first processor 2240 may also control deactivation of the plurality of sensors when receiving off information for the sleep management mode from the input interface 2210.

The first processor 2240 checks the current time, and may control activation of the plurality of sensors when the current time is determined as a bedtime.

The first processor 2240 checks the current time, and may control deactivation of the plurality of sensors when the current time is not determined as a sleeping time.

The sleeping time may be hours between the bedtime and an awake time.

The first processor 2240 may recognize one or more biometric signals based on information received from the plurality of sensors, unite the recognized one or more biometric signals, and recognize sleep state information of the user based on the united information.

The biometric signal may include breathing, heart rate, eye movements, body temperature, oxygen saturation, brain waves, etc.

The configuration of the first processor 2240 for recognizing the biometric signal will now be described in more detail.

The first processor 2240 may recognize a movement of the user based on an image received from the image sensor among the plurality of sensors, and recognize an amount of breathing of the user based on the recognized movement of the user. The first processor 2240 may recognize an amount of breathing for a preset first reference time.

The first processor 2240 may recognize an upper body image of the user from the received image, recognize a movement of the upper body based on the upper body image, and recognize an amount of breathing of the user based on the recognized movement of the upper body.

The first processor 2240 may recognize the upper body image of the user in real time, and determine an image acquisition time for the recognized upper body image.

The first processor 2240 may recognize a first upper body image related to inhalation and a second upper body image related to exhalation based on the upper body image, check a first time at which the first upper body image is obtained and a second time at which the second upper body image is obtained, and recognize an amount of breathing of the user based on the first time and the second time.

The first processor 2240 may also recognize the user's heart rate based on the image. The first processor 2240 may recognize a heart rate during a preset second reference time.

The first processor 2240 may recognize a breathing sound corresponding to the user's breathing among sounds received from the microphone, and may recognize an amount of breathing of the user based on the recognized breathing sound.

The first processor 2240 may recognize the breathing sound of the user in real time and check a time at which the breathing sound is received.

The first processor 2240 may identify the recognized breathing sound as inhalation sound and exhalation sound, check a third time for which the inhalation sound is obtained and a fourth time for which the exhalation sound is obtained, and recognize an amount of breathing of the user based on the third and fourth times.

The first processor 2240 may recognize a movement of the user based on radio wave information received from a radar sensor, and recognize the user's heart rate and amount of breathing based on the recognized movement of the user.

When the sensor is the radar sensor, the first processor 2240 may obtain information about a distance from the radar sensor to the user's body and information about the direction based on a phase difference (or time difference) between a transmission radio wave transmitted from the radar sensor and a reflective radio wave received by the radar sensor, and recognize the user's body based on the obtained distance information and direction information.

The first processor 2240 may obtain a body image by imaging the user's body based on information about a distance to each portion of the user's body in recognizing the user's body through the radar sensor.

The first processor 2240 may identify an upper body part and a lower body part in the user's body image based on the body image.

The first processor 2240 may identify the upper body part as a head portion, a chest portion, an abdominal portion and arm portions.

The first processor 2240 may recognize an amount of the user's breathing based on information about a distance to the chest portion or the abdominal portion of the body image, and recognize the user's heart rate based on information about a distance to the chest portion or the head portion of the body image.

The first processor 2240 may recognize a biometric signal based on information detected by at least one of the image sensor, the microphone and the radar sensor.

When the sensor is a pressure sensor, the first processor 2240 may recognize a movement of the user based on pressure information received from the pressure sensor, and recognize an amount of breathing of the user based on the recognized movement of the user. The amount of breathing may be one during the preset first reference time.

The user's movement may correspond to the upper body's movement due to the user's breathing.

The first processor 2240 may recognize a pressure change based on pressure information received from the pressure sensor, check a fifth time for which pressure increases and a sixth time for which pressure decreases based on the recognized pressure change, and recognize an amount of breathing based on the fifth and sixth times. The pressure information may be a pressure value or a resistance value corresponding to the pressure applied by the user.

When the sensor is the pressure sensor, the first processor 2240 may also recognize the user's heart rate based on the pressure information received from the pressure sensor. The heart rate may be one during the preset second reference time.

When the sensor is the temperature sensor, the first processor 2240 may recognize the user's body temperature based on the temperature information received from the temperature sensor.

When the sensor is the oxygen saturation sensor, the first processor 2240 may recognize an amount of oxygen in the user's blood (i.e., oxygen in blood or an oxygen saturation level) based on oxygen saturation information received from the oxygen saturation sensor.

When the sensor is the heart rate sensor, the first processor 2240 may recognize a heart rate based on the heart rate information detected by the heart rate sensor.

The first processor 2240 may recognize the recognized heart rate of the user, the recognized body temperature of the user, and the recognized oxygen saturation of the user as biometric signals of the user.

The first processor 2240 may recognize a biometric signal based on at least one of image information of the image sensor, sound information of the microphone, pressure information of the pressure sensor, radar information of the radar sensor, body temperature detected by the temperature sensor, a heart rate detected by the heart rate sensor, and oxygen saturation detected by the oxygen saturation sensor, and recognize sleep state information of the user based on the recognized biometric signal.

The first processor 2240 may recognize the biometric signal of the user by learning at least two pieces of the detection information of the plurality of sensors 2100.

The first processor 2240 may recognize the user's sleep state information by learning recognition of the user's biometric signal.

For example, the first processor 2240 may recognize a biometric signal by learning the pre-stored biometric signal, image information of the image sensor, sound information of the microphone, pressure information of the pressure sensor, radar information of the radar sensor, body temperature detected by the temperature sensor, heart rate detected by the heart rate sensor, and oxygen saturation detected by the oxygen saturation sensor, and recognize sleep state information of the user by learning the recognized biometric signals.

The first processor 2240 may determine whether the user is at the pre-sleep stage, the sleeping stage or the post-sleep stage based on the recognized sleep state information of the user.

The pre-sleep stage may include the sleep preparation stage.

The sleeping stage may include such sleeping stages as the NREM sleep stage and the REM sleep stage.

The post-sleep stage may include the awake stage.

The first processor 2240 may determine whether the user is at the sleep preparation stage, the sleeping stage, or the awake stage.

The first processor 2240 may determine the user's sleep stage as stage 1, 2, 3 or 4 of the NREM sleep or the REM sleep stage based on the recognized sleep state information.

### Stage Distinction

The first processor 2240 may recognize the user's heart rate based on at least one of a heart rate obtained by an image of the image sensor, a heart rate obtained by the pressure sensor, and a heart rate obtained by the radar sensor, and determine the user's sleep stage as stage 1, 2, 3 or 4 of the NREM sleep or the REM sleep stage based on the recognized heart rate.

The first processor 2240 may determine the user's stage as the awake stage when the recognized heart rate of the user exceeds a first reference heart rate, determine the user's sleep stage as the stage 1 of the NREM sleep stage when the recognized heart rate of the user is equal to or smaller than the first reference heart rate and exceeds a second reference heart rate, determine the user's sleep stage as the stage 2 of the NREM sleep stage when the recognized heart rate of the user is equal to or smaller than the second reference heart rate and exceeds a third reference heart rate, and determine the user's sleep stage as the stage 3 of the NREM sleep stage when the recognized heart rate of the user is equal to or smaller than the third reference heart rate.

The second reference heart rate may be smaller than the first reference heart rate and greater than the third reference heart rate.

The awake stage may be included in the sleep preparation stage.

The first processor 2240 may recognize an amount of breathing of the user based on an amount of breathing obtained by an image of the image sensor, an amount of breathing obtained by the pressure sensor, and an amount of breathing obtained by the radar sensor, and determine the user's sleep stage as stage 1, 2, 3 or 4 of the NREM sleep or the REM sleep stage based on recognized amount of breathing.

The first processor 2240 may determine the user's sleep stage as the awake stage when the recognized amount of breathing of the user exceeds a first reference amount of breathing, determine the user's sleep stage as the stage 1 of the NREM sleep stage when the recognized amount of breathing of the user is equal to or smaller than the first reference amount of breathing and exceeds a second reference amount of breathing, determine the user's sleep stage as the stage 2 of the NREM sleep stage when the recognized amount of breathing of the user is equal to or smaller than the second reference amount of breathing and exceeds a third reference amount of breathing, and determine the user's sleep stage as the stage 3 of the NREM sleep stage when the recognized amount of breathing of the user is equal to or smaller than the third reference amount of breathing.

The second reference amount of breathing may be smaller than the first reference amount of breathing and greater than the third reference amount of breathing.

When receiving temperature information from the temperature sensor, the first processor 2240 may recognize body temperature of the user based on the temperature information received from the temperature sensor, and determine the user's sleep stage as the stage 2 of the NREM sleep stage when the recognized body temperature is equal to or lower than a reference body temperature.

The first processor 2240 may determine the user's current stage as the sleep preparation stage when the user's biometric signal is recognized by a sensor arranged at the bed.

The sensor arranged at the bed may include at least one of the pressure sensor, the temperature sensor, the radar sensor, the acceleration sensor, the image sensor and the microphone.

When the stages 1, 2, 3 and 4 of the NREM sleep stage are recognized or the REM sleep stage is recognized based on the biometric signal, the first processor 2240 may determine that the current stage of the user is the sleeping stage.

When the biometric signal corresponding to the stage 3 of the NREM sleep stage is not recognized but biometric signals of repeating the stages 1 and 2 of the NREM sleep stage and the REM sleep stage are recognized, the first processor 2240 may determine whether the current time belongs to an awake time range, and determine that the current stage of the user is at the awake stage when the current time is determined as belonging to the awake time range.

When the biometric signal corresponding to the stage 3 or 4 of the NREM sleep stage is not recognized but biometric signals of repeating the stages 1 and 2 of the NREM sleep stage and the REM sleep stage are recognized, the first processor 2240 may determine that the current stage of the user is at the sleeping stage when the current time is not determined as belonging to the awake time range.

### Control of Electronic Device

When the sleep management mode is on, the first processor 2240 communicates with pre-registered electronic devices and recognizes identification information of electronic devices available for connection through the communication.

When determining that the current stage of the user is the sleep preparation stage, the first processor 2240 may communicate with the pre-registered electronic devices and recognize identification information of electronic devices available for connection through the communication.

The first processor 2240 may transmit, to at least one electronic device, an on command or an off command to control the at least one electronic device to correspond to the current stage of the user.

The first processor 2240 may transmit, to at least one electronic device, target value information to control operation of the at least one electronic device to correspond to the current stage of the user.

The on command, off command and target value information may be the control information for the electronic device.

When the current stage of the user is determined as the sleep preparation stage, the first processor 2240 may check the control information for the electronic device for the sleep preparation stage among information stored in the first memory 2250, and control an operation of the electronic device based on the control information for the electronic device.

To control the electronic device at the sleep preparation stage, the first processor 2240 may generate a first control command for the electronic device based on the control information for the electronic device and transmit the first control command to the electronic device.

When there are multiple electronic devices to be controlled, the first control command may be different for each electronic device.

When the electronic devices available to communicate with the hub device are an automatic curtain open/close device, an air conditioner, a humidifier, a TV, a soundbar and a lighting device, in response to the sleep preparation stage, the first processor 2240 may transmit, to the automatic curtain open/close device, a command to close the automatic curtain, transmit, to the air conditioner, a temperature control command for controlling the room temperature, transmit, to the humidifier, a humidity control command for controlling the room humidity, transmit, to the TV, a command to turn off the TV, transmit, to the soundbar, a play command for playing a sleep inducing content, and transmit, to the lighting device, a light control command for controlling light intensity of the room.

When the current stage of the user is determined as the sleeping stage, the first processor 2240 may check the control information for the electronic device for the sleeping stage among the information stored in the first memory 2250, and control an operation of the electronic device based on the control information for the electronic device.

To control the electronic device at the sleeping stage, the first processor 2240 may generate a second control command for the electronic device based on the control information for the electronic device and transmit the second control command to the electronic device.

When there are multiple electronic devices to be controlled, the second control command may be different for each electronic device.

When the electronic devices available to communicate with the hub device are an automatic curtain open/close device, an air conditioner, a humidifier, a TV, a soundbar and a lighting device, in response to the sleeping stage, the first processor 2240 may transmit, to the automatic curtain open/close device, a command to close the automatic curtain, transmit, to the air conditioner, a temperature control command for controlling the room temperature, transmit, to the humidifier, a humidity control command for controlling the room humidity, transmit, to the TV, a command to turn off the TV, transmit, to the soundbar, a command to turn off the sleep inducing content, and transmit, to the lighting device, a light off command.

When the current stage of the user is determined as the awake stage, the first processor 2240 may check the control information for the electronic device for the awake stage among the information stored in the first memory 2250, and control an operation of the electronic device based on the control information for the electronic device.

To control the electronic device at the sleeping stage, the first processor 2240 may generate a third control command for the electronic device based on the control information for the electronic device and transmit the third control command to the electronic device.

When there are multiple electronic devices to be controlled, the third control command may be different for each electronic device.

When the electronic devices available to communicate with the hub device are an automatic curtain open/close device, an air conditioner, a humidifier, a TV, a soundbar and a lighting device, in response to the awake stage, the first processor 2240 may transmit, to the automatic curtain open/close device, a command to open the automatic curtain, transmit, to the air conditioner, a temperature control off command to stop controlling the room temperature, transmit, to the humidifier, a humidity control off command to stop controlling the room humidity, transmit, to the TV, a play command to play contents, transmit, to the soundbar, a command to turn off an audio related content, and transmit, to the lighting device, a light control command.

When a light intensity of the room is set for each stage, the first processor 2240 may check a first light intensity for the sleep preparation stage and control the lighting device to reach the first light intensity in response to the determining as the sleep preparation stage, check a second light intensity for the sleeping stage and control the lighting device to reach the second light intensity in response to the determining as the sleeping stage, and check a third light intensity for the awake stage and control the lighting device to reach the third light intensity in response to the determining as the awake stage.

When a light intensity sensor is provided, it is possible to control on or off of the lighting device based on the light intensity of the room. For example, the first processor 2240 may check the light intensity detected by the light intensity sensor in response to the determining as the awake stage, generate a light on command when the light intensity is less than a reference light intensity, and transmit the light on command to the lighting device.

When the electronic device is a TV or a soundbar, the first processor 2240 may reduce the volume of the TV or soundbar to a preset first volume in response to the determining as the sleep preparation stage, and output the volume of the TV or soundbar at a preset second volume in response to the determining as the awake stage.

The first volume may be lower than the second volume.

When the electronic device is a TV or a projector, the first processor 2240 may reduce the brightness of the TV or soundbar to a preset first brightness in response to the determining as the sleep preparation stage, and output the brightness of the TV or projector at a preset second brightness.

The first brightness may be lower than the second brightness.

When the electronic device is a TV, the first processor 2240 may output a video and audio corresponding to a preset channel in response to the determining as the awake stage.

When the electronic device is a TV or projector, the first processor 2240 may determine whether it is possible to communicate with the user device, receive the user's schedule information from the user device when it is determined that communication with the user device is possible, and control the received schedule information to be displayed in response to the determining as the awake stage.

The first processor 2240 may also receive control information for the electronic devices for each stage of the user from the user device 2300b and store the control information for the electronic devices 2300 for each stage of the user.

### User Identification

The first processor 2240 may identify a user based on an image obtained by the image sensor and a pre-stored user image, and identify a user based on a voice obtained by the microphone and a pre-stored voice.

The first processor 2240 may identify a user based on user identity information received at the input interface 2210 and pre-stored identity information for each user.

The first processor 2240 may identify a user based on voice information received at the microphone and pre-stored voice information for each user.

The first processor 2240 may check control information for one or more electronic devices for each stage corresponding to the identified user's identity information, determine the user's current stage based on biometric signals detected by the plurality of sensors, and control operations of the one or more electronic devices based on the determined user's current stage.

The first processor 2240 may also determine priorities of multiple identified users when it is determined that there are the multiple users, determine identity information of a user having the highest of the determined priorities, obtain control information for the one or more electronic devices for each stage corresponding to the identity information of the user, and control the operation of the one or more electronic devices based on the obtained control information for the one or more electronic devices for each stage.

The first processor 2240 may also receive priority information of the multiple users from the user device 2300b and store the received priority information of the multiple users.

### Recognition of Room and Electronic Device

The first processor 2240 may also communicate with the electronic devices arranged at home and obtain identification information and location information of the electronic devices through the communication with the electronic devices.

The first processor 2240 may also obtain location information of the electronic devices based on received signal strength during the communication with the electronic devices, or receive the location information from the electronic devices.

The first processor 2240 may obtain identification information of a room where each electronic device is arranged, based on room identification information, room location information or the location information of the electronic devices.

The first processor 2240 may store the identification information of the electronic device arranged in each room by matching it with the identification information of the room.

The location information and identification information of the room at home may be configured by the user through the input interface.

The first processor 2240 may match and store the room identification information for each room and the user identity information.

The user identity information matched with the room identification information may be configured by the user through the input interface.

The first processor 2240 may also receive, from the user device 2300b, identification information of the electronic device for each room and identification information of a user for each room, and store the received identification information of the electronic device for each room and identification information of the user for each room.

The first processor 2240 determines a user's current stage for each room based on biometric signals from sensors arranged for each room.

The first processor 2240 may identify a room where a user is at the sleep preparation stage, check the identification information of electronic devices arranged in the identified room, control operations of the electronic devices based on the identification information of the electronic devices, make a change in control of the operations of the electronic devices in response to the user in the identified room being at the sleeping stage, and make a change in control of the operations of the electronic devices in response to the user in the identified room being at the awake stage.

When the control information for the electronic device before a change in stage is the same as the control information for the electronic device after the change in stage, the first processor 2240 may control the operation state of the electronic device to be maintained in response to the change in the user's stage.

The first processor 2240 may avoid disturbance to entrance into a light sleep stage by controlling operations of the electronic devices based on the user's stage related to sleep, and induce to enter into a deep sleep stage.

The first processor 2240 may prevent an increase in power consumption by controlling some of the electronic devices to be turned off based on the user's stage related to sleep being determined as the sleeping stage. That is, according to an embodiment, power consumed by the electronic device may be reduced.

The first memory 2250 may store the identification information of the electronic devices available for communication with the hub device.

The first memory 2250 may store the control information for the electronic devices for each stage.

The first memory 2250 may store time information corresponding to an awake time range, and

time information about a first reference time to check the amount of breathing and a second reference time to check the heart rate.

The first memory 2250 may store reference information to determine a sleep stage.

The reference information may include first, second and third reference amounts of breathing, first, second and third reference heart rates and a reference body temperature.

The reference information may include a reference eye movement rate, reference oxygen saturation, and a reference ECG.

The first memory 2250 may store identification information of each room, and identification information of electronic devices matched with the identification information of the room.

The first memory 2250 may store identity information of the user matched with the identification information of each room.

When there are multiple users, the first memory 2250 may store control information for the electronic devices for each stage corresponding to each user.

When there are multiple users, the first memory 2250 may store priority information of the users.

The first memory 2250 and the first processor 2240 may be implemented in separate chips. Alternatively, the first memory 2250 and the first processor 2240 may be implemented in a single chip.

The first memory 2250 may be implemented with at least one of a non-volatile memory device, such as cache, read only memory (ROM), programmable ROM (PROM), erasable programmable ROM (EPROM), electrically erasable programmable ROM (EEPROM), a volatile memory device, such as random access memory (RAM), or a storage medium, such as hard disk drive (HDD) or compact disk (CD) ROM, without being limited thereto.

Functions related to AI according to embodiments of the disclosure are operated through the first memory 2250 and the first processor 2240. The first processor 2240 may include one or more processors. The one or more first processors 2240 may include a universal processor such as a central processing unit (CPU), an application processor (AP), a digital signal processor (DSP), etc., a graphic processing unit (GPU), a vision processing unit (VPU), etc., or a dedicated artificial intelligence (AI) processor such as a neural processing unit (NPU).

The one or more first processors 2240 may control processing of input data according to a predefined operation rule or an AI model stored in the first memory 2250. When the one or more processors are the dedicated AI processors, they may be designed in a hardware structure that is specific to dealing with a particular AI model.

The predefined operation rule or the AI model may be made by learning. A predefined operation rule or an AI model being made by learning refers to the predefined operation rule or the AI model established to perform a desired feature (or an object) being made when a basic AI model is trained by a learning algorithm with a lot of training data. Such learning may be performed by a device itself in which AI is performed according to the disclosure, or by a separate server and/or system. Examples of the learning algorithm may include supervised learning, unsupervised learning, semi-supervised learning, or reinforcement learning, without being limited thereto.

The AI model may include a plurality of neural network layers. Each of the plurality of neural network layers may have a plurality of weight values, and perform neural network operation through operation between an operation result of the previous layer and the plurality of weight values. The plurality of weight values owned by the plurality of neural network layers may be optimized by learning results of the AI model. For example, the plurality of weight values may be updated to reduce or minimize a loss value or a cost value obtained by the AI model during a training procedure. An artificial neural network may include, for example, a convolutional neural network (CNN), a deep neural network (DNN), a recurrent neural network (RNN), a restricted Boltzmann machine (RBM), a deep belief network (DBN), a bidirectional recurrent deep neural network (BRDNN), or a deep Q-network, without being limited thereto.

The electronic device 2300 may include a second communicator 2310, a second processor 2320, a second memory 2330 and a load 2340.

The second communicator 2310 may include one or more components that enable communication between components in the electronic device 2300.

The second communicator 2310 may include one or more components that enable communication with an external device, for example, at least one of a short-range communication module, a wired communication module, and a wireless communication module. The external device may include the hub device and the server device.

The second communicator 2310 may include both the wired network and the wireless network.

The second processor 2320 controls general operation of the electronic device 2300. There may be one or more second processors 2320. In other words, there may be at least one second processor 2320.

The second processor 2320 may use data stored in the second memory 2330 to perform control related to sleep management. The second processor 2320 may use data stored in the second memory 2330 to control operation of the electronic device 2300.

The second processor 2320 may control the second communicator to communicate with the hub device 2200.

The second processor 2320 may transmit the identification information of the electronic device 2300 to the hub device 2200.

The second processor 2320 may transmit the identification information of the electronic device 2300 to the user device 2300b.

The second processor 2320 controls one or more functions based on a control command received from the hub device 2200. For example, the second processor 2320 may control the load 2340 to perform the one or more functions.

The second processor 2320 may control operation of the load based on a first control command when receiving the first control command.

The second processor 2320 may control operation of the load based on a second control command when receiving the second control command.

The second processor 2320 may control operation of the load based on a third control command when receiving the third control command.

On receiving the first control command, the second processor 2320 may determine that the user is at the sleep preparation stage, check control information corresponding to the sleep preparation stage, and control operation of the load 2340 based on the control information.

On receiving the second control command, the second processor 2320 may determine that the user is at the sleeping stage, check control information corresponding to the sleeping stage, and control operation of the load 2340 based on the control information.

On receiving the third control command, the second processor 2320 may determine that the user is at the awake stage, check control information corresponding to the awake stage, and control operation of the load 2340 based on the control information.

When receiving stage information corresponding to the user's sleep state from the hub device 2200, the second processor 2320 may also control one or more functions based on the received stage information. In this case, the second processor 2320 may store control information for each stage. The control information of each stage may be configured by the user.

On receiving stage information corresponding to the sleep preparation stage from the hub device 2200, the second processor 2320 may check control information corresponding to the sleep preparation stage, and control operation of the load 2340 based on the control information.

On receiving stage information corresponding to the sleeping stage from the hub device 2200, the second processor 2320 may check control information corresponding to the sleeping stage, and control operation of the load 2340 based on the control information.

On receiving stage information corresponding to the awake stage from the hub device 2200, the second processor 2320 may check control information corresponding to the awake stage, and control operation of the load 2340 based on the control information.

The second memory 2330 may store the identification information of the electronic device.

The identification information of the electronic device may include a device name, a model name, etc., of the electronic device.

The second memory 2330 may store the control information for each stage.

The second memory 2330 may store control information corresponding to each of the first, second and third control commands.

The second memory 2330 may store stage information corresponding to each of the first, second and third control commands, and store control information corresponding to the stage information.

The second memory 2330 and the second processor 2320 may be implemented in separate chips. Alternatively, the second memory 2330 and the second processor 2320 may be implemented in a single chip.

The second memory 2330 may be implemented with at least one of a non-volatile memory device, such as cache, ROM, programmable ROM (PROM), erasable programmable ROM (EPROM), electrically erasable programmable ROM (EEPROM), a volatile memory device, such as random access memory (RAM), or a storage medium, such as hard disk drive (HDD) or compact disk (CD) ROM, without being limited thereto.

The load 2340 may be a device for performing a function that may be executed in the electronic device.

The load 2340 may operate in response to a control command of the second processor 2320.

When the electronic device is a TV, the load 2340 may be a display and a speaker.

When the electronic device is a lighting device, the load 2340 may be a lamp.

When the electronic device is an air conditioner, the load 2340 may include a compressor, an air blowing fan, and various valves.

When the electronic device is an automatic curtain open/close device, the load 2340 may be a motor.

When the electronic device is a humidifier, the load 2340 may be an ultrasonic fan or heater depending on the dehumidification method.

When the electronic device is a soundbar, the load 2340 may be a speaker.

When the electronic device is a projector, the load 2340 may be laser and a speaker

When the electronic device is a TV, the TV may turn down the volume or reduce screen brightness based on the sleep preparation stage.

Furthermore, the TV may be powered off based on the sleeping stage.

The TV may be powered on based on the awake stage, may output an image on a channel set by the user, and control audio output based on the volume set by the user.

When the electronic device is a lighting device, the lighting device may reduce its brightness based on the sleep preparation stage, may be powered off based on the sleeping stage, and powered on based on the awake stage.

When the electronic device is an automatic curtain open/close device, the automatic curtain open/close device may be controlled to close the automatic curtain based on the sleep preparation stage and the sleeping stage, and may be controlled to open the automatic curtain based on the awake stage.

When the electronic device is an air conditioner, the air conditioner may control the compressor and the air blowing fan based on the sleep preparation stage to control the air conditioning environment of the room to have a target temperature, target wind direction and target airflow set by the user, control the compressor and the air blowing fan based on the sleeping stage to control the air conditioning environment of the room to have a target temperature, target wind direction and target airflow set by the user, and may be powered off to stop controlling the air conditioning environment of the room based on the awake stage.

The user device 2300b may receive a control command corresponding to the user's current stage from the hub device 2200, and control the display and the speaker based on the received control command.

The user device 2300b may control brightness of the display, turn on the display or turn off the display based on the sleep preparation stage, sleeping stage and awake stage.

The user device 2300b may control the volume of the speaker, turn on the speaker or turn off the speaker based on the sleep preparation stage, sleeping stage and awake stage.

The user device 2300b may receive sleep management information of the sleep management system through a user input, and transmit the received sleep management information to the hub device 2200.

The sleep management information may include information about the user's awake time range, on/off information for each electronic device, and target control value information for each electronic device.

The user device 2300b may transmit the sleep management information for each user at home to the hub device 2200.

The user device 2300b may transmit the sleep management information for each user at home to the hub device 2200 by matching the identity information of the user and the identification information of the room.

The user device 2300b may receive control information for the electronic devices for each stage, and match and transmit, to the hub device 2200, the control information for the electronic devices of each stage with the identification information of the electronic devices.

The user device 2300b may run an application of a platform for sleep management that provides a service to manage the user's sleep. The application may be an application program for providing the sleep management service.

The user device 2300b may control the application for providing the sleep management service to be downloaded, setup and executed.

The user device 2300b may communicate with a server for sleep management, a web server for a website, an application server that provides a sleep monitoring service and a database server.

The user device 2300b may communicate with the sensor 2100 and at least one electronic device, receive a biometric signal from the sensor 2100, determine the user's current stage based on the received biometric signal, generate a control command based on the determined current stage, and transmit the generated control command to the at least one electronic device. In this case, the hub device may be omitted from the sleep management system.

One of the plurality of electronic devices may run the application of the platform for sleep management that provides a service to manage the user's sleep.

The electronic device may communicate with the sensor 2100 and the other electronic devices, receive a biometric signal from the sensor, determine the user's current stage based on the received biometric signal, generate a control command based on the determined current stage, and transmit the generated control command to the other electronic devices. In this case, the hub device may be omitted from the sleep management system.

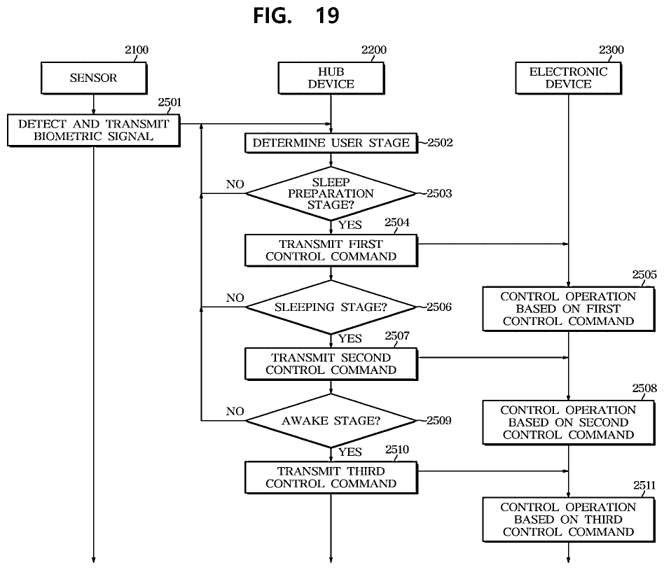

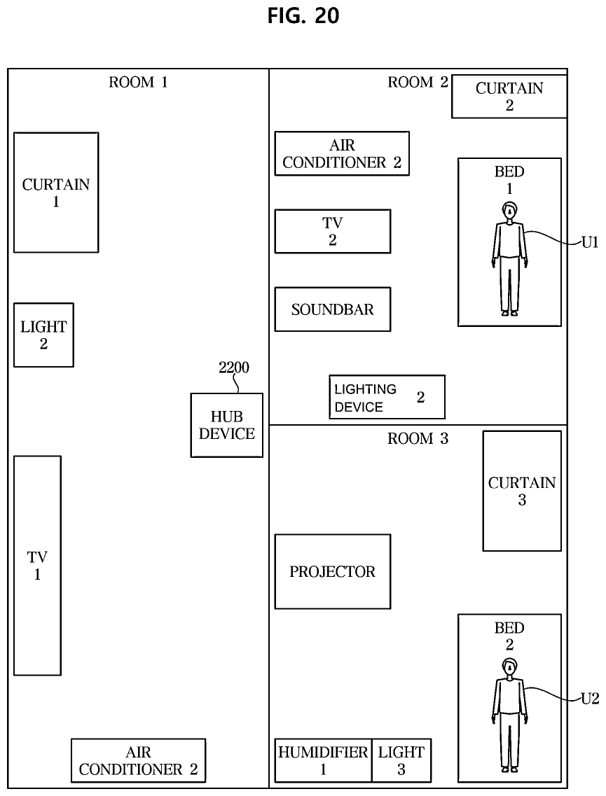

FIG. 19 is a control flowchart of a sleep management system according to an embodiment, which will be described with reference to FIGS. 20 to 24.

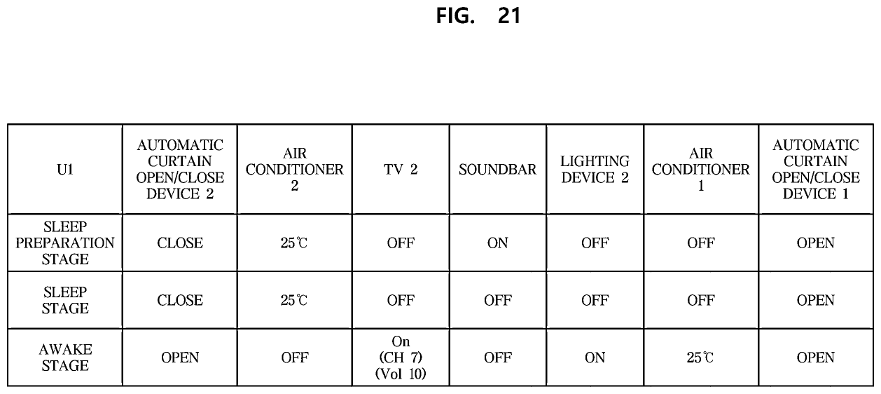

FIG. 20 illustrates layouts of electronic devices at home managed by a sleep management system, according to another embodiment, and FIG. 21 illustrates control information of electronic devices for each stage configured by a sleep management system, according to an embodiment.

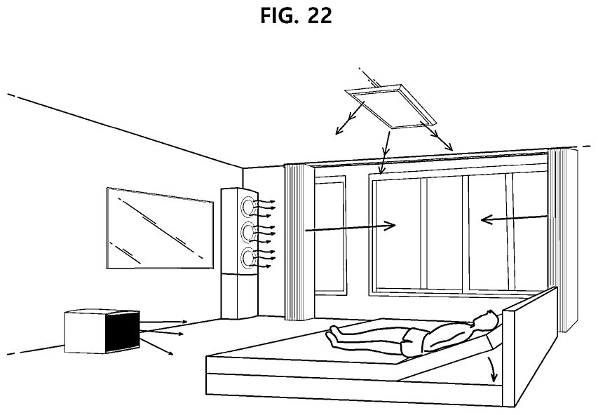

FIGS. 22, 23 and 24 illustrates sleep environments of a room managed by a sleep management system, according to one or more embodiments.

There may be one or more sensors 2100. The sensor 2100 may obtain information about the user or detect a biometric signal of the user, in 2501.

The hub device 2200 may recognize the user's biometric signal based on the information about the user received from the sensor, and recognize the user's sleep state information based on the recognized biometric signal.

For example, the hub device 2200 may recognize at least one of the heart rate and the amount of breathing of the user based on an image received from the image sensor, recognize the amount of breathing of the user based on sound received from the microphone, recognize a movement of the user based on radio wave information received from the radar sensor and recognize at least one of the heart rate and amount of breathing of the user based on the recognized movement of the user, and recognize at least one of the heart rate and amount of breathing of the user based on pressure information received from the pressure sensor.

The hub device 2200 may recognize sleep state information of the user based on a biometric signal received from the sensor.

When multiple biometric signals are obtained or received, the hub device 2200 may unite the multiple biometric signals and recognize the sleep state information of the user based on the united information.

The biometric signals may include an amount of breathing, a heart rate, body temperature, eye movement, oxygen saturation and an ECG of the user.

The hub device 2200 may determine whether the user is at the sleep preparation stage, the sleeping stage or the awake stage based on the sleep state information of the user.

The hub device 2200 may determine the user's current stage, in 2502.

The sleeping stage may include a light sleep stage and a deep sleep stage of the NREM sleep stage, and further include the REM sleep stage.

The NREM sleep stage may include stages 1, 2, 3 and 4.

A specific configuration for determining the user's current stage will now be described.

The hub device 2200 may determine the user's stage as the awake stage when the recognized heart rate of the user exceeds a first reference heart rate, determine the user's sleep stage as the stage 1 of the NREM sleep stage when the recognized heart rate of the user is equal to or smaller than the first reference heart rate and exceeds a second reference heart rate, determine the user's sleep stage as the stage 2 of the NREM sleep stage when the recognized heart rate of the user is equal to or smaller than the second reference heart rate and exceeds a third reference heart rate, and determine the user's sleep stage as the stage 3 of the NREM sleep stage when the recognized heart rate of the user is equal to or smaller than the third reference heart rate.

The second reference heart rate may be smaller than the first reference heart rate and greater than the third reference heart rate.

The awake stage may be included in the sleep preparation stage.

The hub device 2200 may determine the user's sleep stage as the awake stage when the recognized amount of breathing of the user exceeds a first reference amount of breathing, determine the user's sleep stage as the stage 1 of the NREM sleep stage when the recognized amount of breathing of the user is equal to or smaller than the first reference amount of breathing and exceeds a second reference amount of breathing, determine the user's sleep stage as the stage 2 of the NREM sleep stage when the recognized amount of breathing of the user is equal to or smaller than the second reference amount of breathing and exceeds a third reference amount of breathing, and determine the user's sleep stage as the stage 3 of the NREM sleep stage when the recognized amount of breathing of the user is equal to or smaller than the third reference amount of breathing.

The second reference amount of breathing may be smaller than the first reference amount of breathing and greater than the third reference amount of breathing.

When receiving temperature information from the temperature sensor, the hub device may recognize body temperature of the user based on the temperature information received from the temperature sensor, and determine the user's sleep stage as the stage 2 of the NREM sleep stage when the recognized body temperature is equal to or lower than a reference body temperature.

The determining as the awake stage may include determining that the user is at the sleep preparation stage.

When the hub device 2200 determines that a user is present in a sleep area based on a biometric signal received from a sensor, the hub device 2200 may determine the user's stage as the sleep preparation stage.

For example, when recognizing a user who lies on a bed in an image obtained by the image sensor, the hub device 2200 may determine that the user's stage is the sleep preparation stage.

When the hub device 2200 recognizes a movement of the user based on radio wave information obtained by the radar sensor and determines that the user's movement corresponds to a movement of the user who lies on a bed, the hub device 2200 may determine that the user's stage as the sleep preparation stage.

When the biometric signal corresponding to the stage 3 of the NREM sleep stage is not recognized but biometric signals of repeating the stages 1 and 2 of the NREM sleep stage and the REM sleep stage are recognized, the hub device 2200 may determine whether the current time belongs to the awake time range.

When the current time is not determined as belonging to the awake time range, the hub device 2200 may determine to maintain the user's sleep stage and maintain the operations of the electronic devices.

When the current time is determined as belonging to the awake time range, the hub device 2200 may determine that the user is at the awake stage.

When the user is determined as being at the sleep preparation stage in 2503, the hub device 2200 transmits a first control command to the electronic device in 2504.

In this case, on receiving the first control command from the hub device, the electronic device may check control information corresponding to the received first control command and control operation of the load based on the control information in 2505.

When the user is determined as being at the sleeping stage in 2506, the hub device 2200 transmits the first control command to the electronic device in 2507.

In this case, on receiving a second control command from the hub device, the electronic device may check control information corresponding to the received second control command and control operation of the load based on the control information in 2508.

When the user is determined as being at the awake stage in 2509, the hub device 2200 transmits a third control command to the electronic device in 2510.

In this case, on receiving the third control command from the hub device, the electronic device may check control information corresponding to the received third control command and control operation of the load based on the control information in 2511.

The hub device may store identification information of an electronic device to be controlled for each stage.

When there are multiple electronic devices to be controlled for each stage, the hub device may transmit a control command to the multiple electronic devices for each stage. In this case, each electronic device may store control information corresponding to the first, second and third control commands.

As shown in FIG. 20, the hub device may store the user's identity information and the electronic device's identification information that are matched to each of the plurality rooms at home.

For example, identification information of electronic devices of a first room Room1, identity information of a user and identification information of electronic devices of a second room Room2, and identity information of a user and identification information of electronic devices of a third room Room3 may be stored.

When a room is a communal space used by multiple users like the first room Room1, identity information of the user may not be stored.

As in FIG. 21, the user may use the input interface of the hub device or the user device to configure control information for the electronic device for each stage.

For example, a first user U1 may configure control information for electronic devices arranged in the second room Room2 used by the first user U1 for each stage.

The first user U1 may configure control information for a second automatic curtain open/close device, control information for a second air conditioner, control information for a second TV, control information for a soundbar, and control information for a second lighting device at the sleep preparation stage.

The first user U1 may configure control information for a second automatic curtain open/close device, control information for a second air conditioner, control information for a second TV, control information for a soundbar, and control information for a second lighting device at the sleeping stage.

The first user U1 may configure control information for a second automatic curtain open/close device, control information for a second air conditioner, control information for a second TV, control information for a soundbar, and control information for a second lighting device at the awake stage.

The first user U1 may also configure control information for all or part of the electronic devices arranged in the first room Room1 shared with a second user U2.

The first user U1 may configure control information for the first air conditioner and the first automatic curtain open/close device at the sleep preparation stage, configure control information for the first air conditioner and the first automatic curtain open/close device at the sleeping stage, and configure control information for the first air conditioner and the first automatic curtain open/close device at the awake stage,

The hub device may control operations of electronic devices arranged in the second room based on a current stage of the first user, and control operations of electronic devices arranged in the third room based on a current stage of the second user.

As shown in FIG. 22, when the first user is determined as being at the sleep preparation stage, the hub device may close a second automatic curtain by transmitting a close command to a second automatic curtain open/close device, maintain the temperature of the second room at 25° C. by transmitting a temperature control command and a target temperature value to a second air conditioner, turn off a second TV by transmitting an off command to the second TV, control the soundbar to output audio corresponding to a sleep inducing content by transmitting a play command to the soundbar, and turn off a light in the second room by transmitting a light off command to a second lighting device.

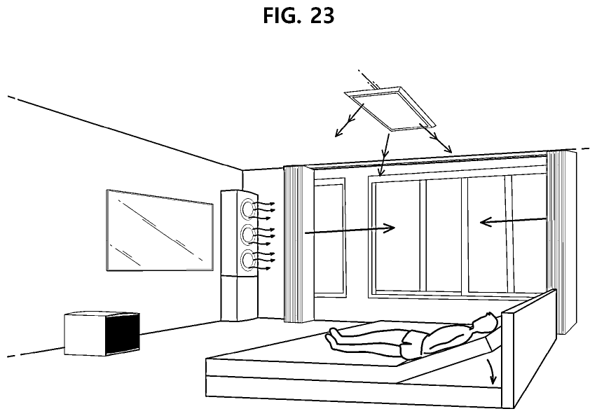

As shown in FIG. 23, when the first user is determined as being at the sleeping stage, the hub device may close the second automatic curtain by transmitting the close command to the second automatic curtain open/close device, maintain the temperature of the second room at 25° C. by transmitting a temperature control command and a target temperature value to the second air conditioner, turn off the second TV by transmitting the off command to the second TV, turn off the soundbar by transmitting an off command to the soundbar, and turn off the light in the second room by transmitting the light off command to a second lighting device.

In controlling the electronic device at the sleeping stage, the hub device may not transmit any control command to an electronic device having the same control information as in the sleep preparation stage.

In controlling the electronic device at the sleeping stage, the hub device may transmit a command to maintain control to an electronic device having the same control information as in the sleep preparation stage.

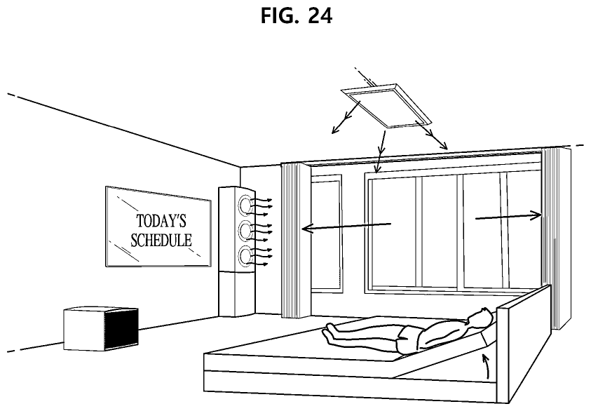

As shown in FIG. 24, when the first user is determined as being at the awake stage, the hub device may open the second automatic curtain by transmitting an open command to the second automatic curtain open/close device, stop controlling the temperature of the second room by transmitting a command to stop temperature control to the second air conditioner, operate the second TV at a set channel and volume by transmitting an on command, channel information and volume information to the second TV, turn off the soundbar by transmitting an off command to the soundbar, and turn on the light in the second room by transmitting a light on command to the second lighting device.

When the first user is determined as being at the awake stage, the hub device may communicate with the user device, receive schedule information of the user from the user device, and display the received schedule information on the TV.

In a case that control information for music play is configured for the awake stage, when the first user is determined as being at the awake stage, the hub device may communicate with the user device, receive the user's song list information from the user device, and play music through the soundbar based on the received song list information.

As such, the electronic devices may be controlled before, during and after the user's sleep, so that the use may take sound sleep and awake feeling refreshed.

One or more embodiments of the disclosure may be implemented in the form of a recording medium for storing instructions to be carried out by a computer. The instructions may be stored in the form of program codes, and when executed by a processor, may generate program modules to perform operations in one or more embodiments of the disclosure. The recording media may correspond to computer-readable recording media.

The computer-readable recording medium includes any type of recording medium having data stored thereon that may be thereafter read by a computer. For example, it may be a read only memory (ROM), a random access memory (RAM), a magnetic tape, a magnetic disk, a flash memory, an optical data storage device, etc.

The computer-readable storage medium may be provided in the form of a non-transitory storage medium. The term ‘non-transitory storage medium’ may mean a tangible device without including a signal, e.g., electromagnetic waves, and may not distinguish between storing data in the storage medium semi-permanently and temporarily. For example, the non-transitory storage medium may include a buffer that temporarily stores data.

In an embodiment of the disclosure, the aforementioned method according to the various embodiments of the disclosure may be provided in a computer program product. The computer program product may be a commercial product that may be traded between a seller and a buyer. The computer program product may be distributed in the form of a recording medium (e.g., a compact disc read only memory (CD-ROM)), through an application store (e.g., Play Store™), directly between two user devices (e.g., smart phones), or online (e.g., downloaded or uploaded). In the case of online distribution, at least part of the computer program product (e.g., a downloadable app) may be at least temporarily stored or arbitrarily created in a recording medium that may be readable to a device such as a server of the manufacturer, a server of the application store, or a relay server.

Several embodiments of the disclosure have been described above, but a person of ordinary skill in the art will understand and appreciate that various modifications can be made without departing from the scope of the disclosure. Thus, it will be apparent to those of ordinary skill in the art that the true scope of technical protection is only defined by the following claims.

What is claimed is:

1. A sleep management system comprising:
at least one sensor configured to detect a biometric signal of a user;
at least one electronic device; and
a hub device configured to:
communicate with the at least one sensor and the at least one electronic device;
identify, based on the biometric signal of the user, a stage as a sleep preparation stage, a sleeping stage, or an awake stage; and
transmit, to the at least one electronic device, a control command to control the at least one electronic device based on the identified stage.

2. The sleep management system of claim 1, wherein the hub device is configured to, based on the identified stage being the sleep preparation stage, transmit at least one of a command to close an automatic curtain, a humidity control command, a temperature control command, or a light-off command.

3. The sleep management system of claim 1, wherein the hub device is configured to, based on the identified stage being the sleeping stage, transmit at least one of a command to turn off a television, a command to turn off a projector, or a command to turn off a soundbar.

4. The sleep management system of claim 1, wherein the hub device is configured to, based on the identified stage being the awake stage, transmit at least one of a command to open an automatic curtain, a command to stop humidity control, a command to stop temperature control, and a light control command.

5. The sleep management system of claim 1, wherein the hub device is configured to, based on the identified stage being the awake stage, communicate with a user device, receive schedule information from the user device, and transmit a command to output the received schedule information.

6. The sleep management system of claim 1, wherein the at least one sensor comprises at least one of an image sensor, a microphone, a radar sensor, a pressure sensor, a temperature sensor, an electrocardiogram (ECG) sensor, an acceleration sensor, or a heart rate sensor,
wherein the at least one electronic device comprises at least one of an automatic curtain open/close device, a television, a projector, a soundbar, a lighting device, an air conditioner, or a humidifier.

7. The sleep management system of claim 6, wherein the hub device comprises an input interface; and
a processor configured to generate a control command for each of the at least one electronic device based on target value information for each of the at least one electronic device received through the input interface.

8. The sleep management system of claim 1, wherein:
the at least one sensor is arranged at a bed placed in a room or a user device, and
the at least one sensor or the user device comprises a communicator for communicating with the hub device.

9. The sleep management system of claim 1, wherein the hub device comprises:
an input interface; and
a processor configured to identify the awake stage based on the biometric signal and awake time information received through the input interface.

10. A method of controlling a sleep management system, the method comprising:
detecting, using at least one sensor, a biometric signal of a user;
identifying, through a hub device based on the biometric signal of the user, a stage as a sleep preparation stage, a sleeping stage or an awake stage; and
transmitting a control command to control at least one electronic device based on the identified stage to the at least one electronic device,
wherein the at least one electronic device is configured to control an operation based on the control command.

11. The method of claim 10, wherein the transmitting of the control command comprises transmitting at least one of a command to close an automatic curtain, a humidity control command, a temperature control command, or a light off command based on the determining as the sleep preparation stage,
wherein the transmitting of the control command comprises, based on the identified stage being the sleep preparation stage, transmitting a command to play a sleep inducing content.

12. The method of claim 10, wherein the transmitting of the control command comprises, based on the identified stage being the sleeping stage, transmitting at least one of a command to turn off a television, a command to turn off a projector, or a command to turn off a soundbar.

13. The method of claim 10, wherein the transmitting of the control command comprises transmitting, based on the identified stage being the awake stage, at least one of a command to open an automatic curtain, a command to stop humidity control, a command to stop temperature control, or a light on command.

14. The method of claim 10, wherein the transmitting of the control command comprises transmitting, based on the identified stage being the awake stage, at least one of a television on command, a channel command and a volume command.

15. The method of claim 10, wherein the transmitting of the control command comprises, based on the identified stage being the awake stage, communicating with a user device, receiving schedule information from the user device, and transmitting a command to output the received schedule information.
